(19) United States
(12) Patent Application Publication
(10) Pub. No.: US 2003/0170871 A1
(43) Pub. Date: Sep. 11, 2003

(54) Alphavirus-based vectors for persistent infection

(75) Inventors: Thomas W. Dubensky JR., Piedmont, CA (US); John M. Polo, Hayward, CA (US); Silvia Perri, Castro Valley, CA (US); Barbara Belli, San Diego, CA (US)

Correspondence Address: ANNE S. DOLLARD, ESQ., CHIRON CORPORATION, INTELLECTUAL PROPERTY - R440, P.O. BOX 8097, EMERYVILLE, CA, 94662-8097, US

(73) Assignee: CHIRON CORPORATION

(21) Appl. No.: 09/841,994

(22) Filed: Apr. 25, 2001

Related U.S. Application Data
(60) Provisional application No. 60/199,579, filed on Apr. 25, 2000.

(51) Int. Cl. A61K 48/00; C12P 21/02; C12N 15/86; C12N 5/06; C12N 7/00; C07H 21/04; A01N 63/00

(52) U.S. Cl. 435/235.1; 424/93.21; 435/325; 435/456; 435/69.1; 536/23.72

(57) ABSTRACT

Isolated nucleic acid molecules are disclosed, comprising an alphavirus nonstructural protein 2 gene which, when operably incorporated into an alphavirus replicon particle, eukaryotic layered vector initiation system, alphavirus vector construct or RNA vector replicon, provides a noncytopathic phenotype or confers the ability to establish persistent replication. Also disclosed are RNA vector replicons, alphavirus vector constructs, alphavirus replicon particles and eukaryotic layered vector initiation systems which contain the above-identified nucleic acid molecules, as well as methods of using such replicons, constructs, particles and eukaryotic layered vector initiation systems for expression of recombinant proteins.

### CROSS REFERENCE TO RELATED APPLICATIONS

This application claims priority to U.S. Provisional Application No. 60/199,579, filed Apr. 25, 2000, which application is incorporated by reference in its entirety.

### REFERENCE TO SEQUENCE LISTING, TABLES OR COMPUTER PROGRAM LISTING

A Sequence Listing in computer readable format is included herewith.

### BACKGROUND OF THE INVENTION

The present invention relates generally to recombinant DNA technology and more specifically, to the development of recombinant alphavirus vectors useful for directing the expression of one or more heterologous gene products in the absence of vector induced cytopathology.

Alphaviruses comprise a set of genetically, structurally, and serologically related arthropod-borne viruses of the Togaviridae family. Twenty-six known viruses and virus subtypes have been classified within the alphavirus genus, including, Sindbis virus, Semliki Forest virus, Ross River virus, and Venezuelan equine encephalitis virus.

Sindbis virus is the prototype member of the Alphavirus genus of the Togaviridae family. Its replication strategy has been well characterized in a variety of cultured cells and serves as a model for other alphaviruses. Briefly, the genome from Sindbis virus (like other alphaviruses) is an approximately 12 kb single-stranded positive-sense RNA molecule which is capped and polyadenylated, and contained within a virus-encoded capsid protein shell. The nucleocapsid is further surrounded by a host-derived lipid envelope into which two viral glycoproteins, E1 and E2, are inserted and anchored to the nucleocapsid. Certain alphaviruses (e.g., SFV) also maintain an additional protein, E3, which is a cleavage product of the E2 precursor protein, PE2.

After virus particle adsorption to target cells, penetration, and uncoating of the nucleocapsid to release viral genomic RNA into the cytoplasm, the replicative process is initiated by translation of the nonstructural proteins (nsPs) from the 5′ two-thirds of the viral genome. The four nsPs (nsP1-nsP4) are translated directly from the genomic RNA template as one of two polyproteins (nsP123 or nsP1234), and processed post-translationally into monomeric units by an active protease in the C-terminal domain nsP2. A leaky opal (UGA) codon present between nsP3 and nsP4 of most alphaviruses accounts for a 10 to 20% abundance of the nsP1234 polyprotein, as compared to the nsP123 polyprotein. Both of the nonstructural polyproteins and their derived monomeric units may participate in the RNA replicative process, which involves binding to the conserved nucleotide sequence elements (CSEs) present at the 5′ and 3′ ends, and a junction region subgenomic promoter located internally in the genome (discussed further below).

The positive strand genomic RNA serves as template for the nsP-catalyzed synthesis of a full-length complementary negative strand. Synthesis of the complementary negative strand is catalyzed after binding of the nsP complex to the 3′ terminal CSE of the positive strand genomic RNA. The negative strand, in turn, serves as template for the synthesis of additional positive strand genomic RNA and an abundantly expressed 26S subgenomic RNA, initiated internally at the junction region promoter. Synthesis of additional positive strand genomic RNA occurs after binding of the nsP complex to the 3′ terminal CSE of the complementary negative strand genomic RNA template. Synthesis of the subgenomic mRNA from the negative strand genomic RNA template, is initiated from the junction region promoter. Thus, the 5′ end and junction region CSEs of the positive strand genomic RNA are functional only after they are transcribed into the negative strand genomic RNA complement (i.e., the 5′ end CSE is functional when it is the 3′ end of the genomic negative stranded complement). The structural proteins (sPs) are translated from the subgenomic 26S RNA, which represents the 3′ one-third of the genome, and like the nsps, are processed post-translationally into the individual proteins.

Several members of the alphavirus genus are being developed as “replicon” expression vectors for in vitro and in vivo use. These alphaviruses include, for example, Sindbis virus (Xiong et al., Science 243:1188-1191, 1989; Dubensky et al., J. Virol. 70:508-519, 1996; Hariharan et al., J. Virol. 72:950-958, 1988; Polo et al., PNAS 96:4598-4603, 1999), Semliki Forest virus (Liljestrom, Bio/Technology 9:1356-1361, 1991; Berglund et al., Nat. Biotech. 16:562-565, 1998), and Venezuelan equine encephalitis virus (Pushko et al., Virology 239:389-401). The use of alphavirus vectors generally has been limited to applications where extended periods of heterologous gene expression is not required because vector-induced inhibition of host cell-directed macromolecular synthesis (i.e., protein or RNA synthesis) begins within a few hours after infection, culminating in eventual cell death.

More recently, Sindbis virus variants and their derived vectors have been described, which display significantly reduced inhibition of host macromolecular synthesis (WO 9738087; WO 9918226; Agapov et al., PNAS 95:12989-12994, 1998; Frolov et al., J. Virol. 73:3854-3865, 1999). In addition, these virus and vector variants show reduced levels of Sindbis RNA, but maintain high level expression of vector encoded heterologous genes. Unfortunately, efficient packaging of these SIN replicon vectors was not observed. The phenotypic changes in the Sindbis virus and vector variants described in these references were attributed to mutation of amino acid residue 726 of nsP2.

The present invention provides novel Sindbis virus and Semliki Forest virus replicon vectors with the desired phenotype of reduced inhibition of host macromolecular synthesis, reduced vector RNA synthesis, high level heterologous gene expression, and in several cases, efficient packaging into alphavirus replicon particles (Perri et al., J. Virol. 74:9802-9807, 2000). The compositions described herein may be used for a variety of applications, including for example, gene delivery in vitro and in vivo, as well as production of recombinant proteins in cultured cells.

### BRIEF SUMMARY OF THE INVENTION

Briefly stated, the present invention provides RNA vector replicons, alphavirus vector constructs, eukaryotic layered vector initiation systems and alphavirus replicon particles which exhibit reduced, delayed, or no inhibition of host cell macromolecular synthesis (e.g., protein or RNA synthesis), thereby permitting the use of these vectors for protein expression, gene delivery and the like, with reduced, delayed, or no development of CPE or cell death. Such vectors may be constructed from a wide variety of alphaviruses (e.g., Semliki Forest virus, Ross River virus, Venezuelan equine encephalitis virus, Sindbis virus), and may be used to express a variety of heterologous proteins (e.g., therapeutic proteins).

Within one aspect of the invention, isolated nucleic acid molecules are provided comprising an altered alphavirus nonstructural protein 2 gene which, when operably incorporated into an alphavirus RNA vector replicon, alphavirus vector construct, alphavirus replicon particle, or eukaryotic layered vector initiation system, increases the time required to reach 50% inhibition of host-cell directed macromolecular synthesis following expression in mammalian cells, as compared to the analogous vector or particle containing a wild-type alphavirus nonstructural protein 2 gene. In addition, it should be understood that when the isolated nucleic acid molecules of the present invention are incorporated into an alphavirus RNA vector replicon, alphavirus vector construct, alphavirus replicon particle, or eukaryotic layered vector initiation system, that they may, within certain embodiments, substantially increase the time required to reach 50% inhibition of host-cell directed macromolecular synthesis, up to and including substantially no detectable inhibition of host-cell directed macromolecular synthesis (over any period of time). Assays suitable for detecting percent inhibition of host-cell directed macromolecular synthesis include, for example, those assays described in this specification.

Within another aspect of the invention, isolated nucleic acid molecules are provided comprising an altered alphavirus nonstructural protein 2 gene which, when operably incorporated into an alphavirus RNA vector replicon, alphavirus vector construct, alphavirus replicon particle, or eukaryotic layered vector initiation system, allows for the persistent replication of said vector or particle, following introduction into a mammalian cell. In addition, such vectors or particles may, within certain embodiments, further comprise and express a heterologous selection marker, such as an antibiotic resistance gene. Representative examples of such antibiotic resistance markers include hygromycin phosphotransferase and neomycin phosphotransferase.

Within other aspects of the invention, isolated nucleic acid molecules are provided comprising an altered alphavirus nonstructural protein 2 gene which, when operably incorporated into an alphavirus replicon particle, alphavirus vector construct, eukaryotic layered vector initiation system, or alphavirus RNA vector replicon, results in a reduced level (e.g., 2-fold, 5-fold, 10-fold, 50-fold, greater than 100-fold) of vector-specific RNA synthesis as compared to the wild-type, and the same or greater level of protein encoded by RNA transcribed from the viral junction region promoter, as compared to the analogous vector or particle containing a wild-type alphavirus nonstructural protein 2 gene. In yet another aspect, the level of heterologous protein expression from RNA transcribed from the viral junction region promoter is also reduced, but the reduction is at least 50% less than the level of reduction for vector-specific RNA synthesis. Representative assays that are standard techniques in the art for quantitating RNA levels include [³H] uridine incorporation or RNA accumulation as detected by Northern blot analysis, as described in the Examples. Representative assays for quantitating protein levels include scanning densitometry, FACS analysis, and various enzymatic assays, as described in the Examples.

In preferred embodiments, the altered alphavirus nonstructural protein 2 gene described above encodes a nonstructural protein 2 with a substitution in or deletion of an amino acid of nsP2 selected from the group consisting of amino acid 1, 10, 469, 472, 713, and 721.

Within another aspect of the present invention, alphavirus vector constructs are provided, comprising a 5′ promoter which initiates synthesis of viral RNA in vitro or in vivo from cDNA, a 5′ sequence which initiates transcription of alphavirus RNA, a nucleic acid molecule which operably encodes all four alphaviral nonstructural proteins including an isolated nucleic acid molecule as described above, an alphavirus subgenomic junction region promoter, an alphavirus RNA polymerase recognition sequence and a 3′ polyadenylate tract. Representative examples of suitable 5′ promoters for synthesis of viral RNA in vivo from an alphavirus vector construct (as well as eukaryotic layered vector initiation system) include for example, RNA polymerase I promoters, RNA polymerase 11 promoters (e.g., HSV-TK, RSV, MOMLV, SV40 and CMV promoter), RNA polymerase III promoters. Within one preferred embodiment, the 5′ promoter is an inducible promoter (e.g., tetracycline inducible promoter). Representative examples of suitable 5′ promoters for synthesis of viral RNA in vitro, from an alphavirus vector construct, include for example, bacteriophage SP6, T7 and T3 promoters.

Within yet other aspects of the present invention, RNA vector replicons capable of translation in a eukaryotic system are provided, comprising a 5′ sequence which initiates transcription of alphavirus RNA, a nucleic acid molecule which operably encodes all four alphaviral nonstructural proteins, including an isolated nucleic acid molecule discussed above, an alphavirus subgenomic junction region promoter, an alphavirus RNA polymerase recognition sequence and a 3′ polyadenylate tract.

Within a related aspect, such alphavirus replicon particles, eukaryotic layered vector initiation systems, RNA vector replicons, or alphavirus vector constructs further comprise a selected heterologous sequence position downstream of and operably linked to the alphavirus subgenomic junction region promoter. Within further aspects of the invention, host cells are provided which contain an alphavirus RNA vector replicon, alphavirus vector construct, or eukaryotic layered vector initiation system, or which have been infected with an alphavirus replicon particle, described herein. Such host cells may be of mammalian or non-mammalian origin. Within additional aspects of the invention, pharmaceutical compositions are provided comprising RNA vector replicons, alphavirus replicon particles, alphavirus vector constructs or eukaryotic layered vector initiation systems as described herein and a pharmaceutically acceptable carrier or diluent.

Within related aspects, the present invention also provides eukaryotic host cells (e.g., vertebrate or non-vertebrate, mammalian or non-mammalian) containing a stably transformed eukaryotic layered vector initiation system or alphavirus vector construct as described above. Within further aspects of the present invention, methods for delivering a selected heterologous sequence to a eukaryotic cell are provided, comprising the step of administering to the eukaryotic cell an alphavirus vector construct, alphavirus RNA vector replicon, alphavirus replicon particle, or a eukaryotic layered vector initiation system as described herein. Within certain embodiments, the alphavirus vector construct, alphavirus RNA vector replicon, alphavirus replicon particle or eukaryotic layered vector initiation system is administered to the cells ex vivo, followed by administration of said cells to a warm-blooded animal. Within other embodiments, the alphavirus vector construct, alphavirus RNA vector replicon, alphavirus replicon particle or eukaryotic layered vector initiation system is administered to the cells in vivo.

Within yet other aspects, methods of making a selected protein are provided, comprising the step of introducing into a eukaryotic host cell an alphavirus vector construct, alphavirus RNA vector replicon, alphavirus replicon particle or eukaryotic layered vector initiation system as described herein, further comprising a gene encoding the selected protein, under conditions and for a time sufficient to permit expression of the selected protein. Within certain embodiments, the host cell is stably transformed with said vector or alphavirus replicon particle.

These and other aspects and embodiments of the invention will become evident upon reference to the following detailed description and attached figures. In addition, various references are set forth herein that describe in more detail certain procedures or compositions (e.g., plasmids, sequences, etc.), and are therefore incorporated by reference in their entirety as if each were individually noted for incorporation.

### BRIEF DESCRIPTION OF THE DRAWINGS

FIG. 1A is a Northern blot of replicon specific RNAs.

FIG. 1B is a graph showing expression that results from replicon variants present in transfected drug resistant cells.

FIG. 2A is a schematic illustration of the mapping of SIN variants.

FIG. 2B is a schematic illustration of the mapping of SFV variants.

FIG. 2C shows SIN and SFV mutations causing the desired phenotype.

FIG. 3A shows subgenomic to genomic RNA ratios of the variants.

FIG. 3B shows the level of heterologous gene expression from the variants.

FIG. 4 is a PCR analysis showing differences in RNA levels.

FIGS. 5A,B,C shows processing of the nonstructural polyprotein.

FIG. 6 shows the sequence of an R17/MS2 translational operator.

FIG. 7 is a schematic illustration of a temperature sensitive recombinant protein expression system using DNA-based alphavirus replicons.

FIG. 8 is a schematic illustration of a producer cell system for the production of alphavirus replicon particles.

### DETAILED DESCRIPTION OF THE INVENTION

### Definition of Terms

The following terms are used throughout the specification. Unless otherwise indicated, these terms are defined as follows:

“Altered alphavirus nonstructural protein 2 gene” refers to an alphavirus nsP2 gene which, when operably incorporated into an alphavirus RNA vector replicon, alphavirus vector construct, alphavirus replicon particle, or eukaryotic layered vector initiation system, produces the desired phenotype (e.g., reduced, delayed or no inhibition of cellular macromolecular synthesis or ability to establish persistent replication). The altered alphavirus nonstructural protein 2 gene should have one or more nucleotide substitutions or deletions that alter the nucleotide sequence from that of the wild-type alphavirus gene, with at least one of said substitutions or deletions at nonstructural protein 2 amino acid residue 1, 10, 469, 472, 713 or 721.

“Genomic RNA” refers to RNA that contains all of the genetic information required to direct its own amplification or self-replication in vivo, within a target cell. To direct its own replication, the RNA molecule may: 1) encode one or more polymerase, replicase, or other proteins which may interact with viral or host cell-derived proteins, nucleic acids or ribonucleoproteins to catalyze the RNA amplification process; and 2) contain cis RNA sequences required for replication, which may be bound during the process of replication by its self-encoded proteins. An alphavirus-derived genomic RNA molecule should contain the following ordered elements: 5′ viral or defective-interfering RNA sequence(s) required in cis for replication, sequences which, when expressed, code for biologically active alphavirus nonstructural proteins (e.g., nsP1, nsP2, nsP3, nsP4), 3′ viral sequences required in cis for replication, and a polyadenylate tract. The alphavirus-derived genomic RNA, including vector replicon RNA, also may contain a viral subgenomic “junction region” promoter. Generally, the term genomic RNA refers to a molecule of positive polarity, or “message” sense, and the genomic RNA may be of length different from that of any known, naturally-occurring alphavirus. In preferred embodiments, the genomic RNA does not contain sequences that encode any alphaviral structural protein(s); rather those sequences are substituted with a heterologous sequence(s).

“Subgenomic RNA” refers to an RNA molecule of a length or size, which is smaller than the genomic RNA from which it was derived. The subgenomic RNA should be transcribed from an internal promoter whose sequences reside within the genomic RNA or its complement. Transcription of the subgenomic RNA usually is mediated by viral-encoded polymerase or transcriptase (e.g., nsP1, 2, 3, or 4). In preferred embodiments, the subgenomic RNA is produced from a vector according to the invention, and encodes or expresses a heterologous gene or sequence.

“Alphavirus vector construct” refers to an assembly which is capable of directing the expression of a sequence(s) or gene(s) of interest. Such vector constructs are comprised of a 5′ sequence which is capable of initiating transcription of an alphavirus RNA (also referred to as 5′ CSE, in background), as well as sequences which, when expressed, code for biologically active alphavirus nonstructural proteins (e.g., nsP1, nsP2, nsP3, nsP4), and an alphavirus RNA polymerase recognition sequence (also referred to as 3′ CSE, in background). In addition, the vector construct should include a viral subgenomic “junction region” promoter that may, in certain embodiments, be modified in order to prevent, increase, or reduce viral transcription of the subgenomic fragment, and also a polyadenylate tract. The vector also may include a 5′ promoter which is capable of initiating the synthesis of viral RNA in vitro or in vivo from cDNA and a heterologous sequence(s) to be expressed.

“Alphavirus RNA vector replicon”, “RNA vector replicon” and “replicon” refers to an RNA molecule which is capable of directing its own amplification or self-replication in vivo, within a target cell. To direct its own amplification, the RNA molecule should encode polymerase(s) necessary to catalyze RNA amplification (e.g., nsP1, 2, 3 or 4) and contain cis RNA sequences required for replication which may be bound by the encoded polymerase(s). An alphavirus-derived RNA vector replicon should contain the following ordered elements: 5′ viral sequences required in cis for replication (also referred to as 5′ CSE, in background), sequences which, when expressed, code for biologically active alphavirus nonstructural proteins (e.g., nsP1, nsP2, nsP3, nsP4), 3′ viral sequences required in cis for replication (also referred to as 3′ CSE, in background), and a polyadenylate tract. The alphavirus-derived RNA vector replicon also may contain a viral subgenomic “junction region” promoter which may, in certain embodiments, be modified in order to prevent, increase, or reduce viral transcription of the subgenomic fragment, and heterologous sequence(s) to be expressed.

“Alphavirus Replicon Particle” or “Recombinant Alphavirus Particle” refers to a virion unit containing an alphavirus RNA vector replicon. Generally, the alphavirus replicon particle comprises one or more alphavirus structural proteins, a lipid envelope and an RNA vector replicon. Preferably, the alphavirus replicon particle contains a nucleocapsid structure that is contained within a host cell-derived lipid bilayer, such as a plasma membrane, in which alphaviral-encoded envelope glycoproteins are embedded. The particle may also contain other components (e.g., targeting elements such as biotin, other viral structural proteins, or other receptor binding ligands) which direct the tropism of the particle from which the alphavirus was derived.

“Structural protein expression cassette” refers to a nucleic acid molecule that directs the synthesis of one or more alphavirus structural proteins. The expression cassette should include a 5′ promoter which is capable of initiating in vivo the synthesis of RNA from cDNA, as well as sequences which, when expressed, code for one or more biologically active alphavirus structural proteins (e.g., C, E3, E2, 6K, El), and a 3′ sequence which controls transcription termination. The expression cassette also may include a 5′ sequence which is capable of initiating transcription of an alphavirus RNA (also referred to as 5′ CSE, in background), a viral subgenomic “junction region” promoter, and an alphavirus RNA polymerase recognition sequence (also referred to as 3′ CSE, in background).

“Stable Transformation” refers to the introduction of a nucleic acid molecule into a living cell, and long-term or permanent maintenance of that nucleic acid molecule in progeny cells through successive cycles of cell division. The nucleic acid molecule may be maintained in any cellular compartment, including, but not limited to, the nucleus, mitochondria, or cytoplasm. In preferred embodiments, the nucleic acid molecule is maintained in the nucleus. Maintenance may be intrachromosomal (integrated) or extrachromosomal, as an episomal event.

“Alphavirus packaging cell line” refers to a cell which contains an alphavirus structural protein expression cassette and which produces alphavirus replicon particles after introduction of an alphavirus vector construct, RNA vector replicon, eukaryotic layered vector initiation system, or alphavirus replicon particle. The parental cell may be of mammalian or non-mammalian origin. Within preferred embodiments, the packaging cell line is stably transformed with the structural protein expression cassette.

“Eukaryotic Layered Vector Initiation System” refers to an assembly that is capable of directing the expression of a sequence(s) or gene(s) of interest. The eukaryotic layered vector initiation system should contain a 5′ promoter which is capable of initiating in vivo (i.e. within a cell) the synthesis of RNA from cDNA, and a nucleic acid vector sequence (e.g., viral vector) which is capable of directing its own replication in a eukaryotic cell and also expressing a heterologous sequence. In certain embodiments, the nucleic acid vector sequence is an alphavirus-derived sequence and is comprised of a 5′ sequence which is capable of initiating transcription of an alphavirus RNA (also referred to as 5′ CSE, in background), as well as sequences which, when expressed, code for biologically active alphavirus nonstructural proteins (e.g., nsP1, nsP2, nsP3, nsP4), and an alphavirus RNA polymerase recognition sequence (also referred to as 3′ CSE, in background). In addition, the vector sequence may include an alphaviral subgenomic junction region promoter which may, in certain embodiments, be modified in order to prevent, increase, or reduce viral transcription of the subgenomic fragment, as well as a polyadenylation sequence. The eukaryotic layered vector initiation system may also contain splice recognition sequences, a catalytic ribozyme processing sequence, a nuclear export signal, and a transcription termination sequence. In certain embodiments, in vivo synthesis of the vector nucleic acid sequence from cDNA may be regulated by the use of an inducible promoter or subgenomic expression may be inducible through the use of translational regulators or modified nonstructural proteins.

Numerous aspects and advantages of the invention will be apparent to those skilled in the art upon consideration of the following detailed description which provides illumination of the practice of the invention.

As noted above, the present invention provides novel alphavirus RNA vector replicons, alphavirus vector constructs, eukaryotic layered vector initiation systems and alphavirus replicon particles that exhibit reduced, delayed, or no inhibition of host cell-directed macromolecular synthesis following introduction into a host cell, as compared to wild-type derived vectors. Also provided are representative examples of heterologous sequences that may be expressed by the alphavirus vectors of the present invention, as well as cell lines containing the alphavirus vectors.

### Sources of Wild-Type Alphavirus

Sequences encoding wild-type alphaviruses suitable for use in preparing the above-described vectors can be readily obtained from naturally occurring sources or from depositories (e.g., the American Type Culture Collection, Rockville, Md.). Representative examples include Ross River virus (ATCC VR-373, ATCC VR-1246), Semliki Forest virus (ATCC VR-67, ATCC VR-1247), Sindbis virus (ATCC VR-68, ATCC VR-1248) and Venezuelan equine encephalitis virus (ATCC VR69, ATCC VR-923, ATCC VR-1250 ATCC VR-1249, ATCC VR-532). In addition, wild-type alphaviruses and their derived vectors may be utilized for comparing the level of host-cell directed macromolecular synthesis in cells infected with or containing the wild-type alphavirus or its derived vectors, with the level of host-cell directed macromolecular synthesis in cells infected with or containing the alphavirus derived vectors of the present invention. Similar reagents may be used for comparing the ability to establish persistent replication in a host cell. For purposes of comparing levels of cellular macromolecular synthesis, the following plasmids may also be utilized as a standard source of wild-type alphavirus stocks. These plasmids include: for Semliki Forest virus, pSP6-SFV4 (Liljestrom et al., J. Virol. 65:4107-4113, 1991); for Venezuelan equine encephalitis virus, pV2000 (Davis et al., Virology 183:20-31, 1991); for Ross River virus, pRR64 (Kuhn et al., Virology 182:430-441, 1991); for Sindbis virus, pTRSB (McKnight et al., J. Virol. 70:1981-1989, 1996); for S.A.AR86 virus, pS55 (Simpson et al., Virology 222:464-469, 1996). Briefly, for these plasmids, virus can be obtained from BHK cells transfected with in vitro transcribed genomic RNA from the plasmids. For Sindbis virus, infectious virus also may be isolated directly from BHK cells transfected with pVGELVIS (Dubensky et al., ibid; ATCC No. 75891) plasmid DNA.

Alphavirus Vector Variants With a Desired Phenotype

Within various embodiments of the present invention, alphavirus vectors and replicon particles are provided, which contain a nsP2 gene with at least one mutation located at amino acid residue 1, 10, 469, 472, 713 or 721. Within one embodiment, nsP2 codon 1 is mutated to another amino acid selected from the group consisting of Arg, Asn, Asp, Asx, Cys, GIn, Glu, Glx, His, lie, Leu, Lys, Met, Phe, Pro, Ser, Thr, Trp, Tyr, Val, or another rare or non-protein amino acid (see, e.g., Lehninger, Biochemistry, Worth Publishers, Inc., N.Y. N.Y., 1975). Within another embodiment, nsP2 codon 10 is mutated to another amino acid selected from the group consisting of Ala, Arg, Asn, Asp, Asx, Cys, Gln, Glu, Glx, Gly, His, Lys, Met, Phe, Pro, Ser, Thr, Trp, Tyr, or another rare or non-protein amino acid. Within another embodiment, nsP2 codon 469 or 472 is mutated to another amino acid selected from the group consisting of Ala, Arg, Asx, Cys, Gin, Glu, Glx, Gly, His, lie, Leu, Lys, Met, Phe, Pro, Ser, Thr, Trp, Tyr, Val, or another rare or non-protein amino acid. Within yet another embodiment, nsP2 codon 713 or 721 is mutated to another amino acid selected from the group consisting of Ala, Arg, Asn, Asp, Asx, Gin, Glu, Glx, Gly, His, Ile, Lys, Met, Phe, Pro, Ser, Thr, Trp, Tyr, Val, or another rare or non-protein amino acid. Alternatively, in other embodiments, relatively conserved regions within which the above-specified amino acids reside may contain an amino acid substitution from the wild-type, instead of, or in addition to those specified. For example, nsP2 amino acids 1-7 are relatively conserved among alphaviruses, with amino acids 3-7 being absolutely conserved among published wild-type strains of Sindbis virus, S.A.AR86 virus, Venezuelan equine encephalitis virus, Ross River virus, and Semliki Forest virus. Amino acids 10-12 show only conservative amino acid differences among the same viruses. Alternatively, the extreme carboxy terminal amino acids of nsPl (e.g., the last 2), which are immediately adjacent to nsP2 amino acid 1 and part of the cleavage recognition site, may contain amino acid changes from wild-type. Within certain embodiments of the invention, the above amino acid codons may be deleted.

### Alphavirus Vector Constructs and Alphavirus RNA Vector Replicons

As noted above, the present invention provides both DNA and RNA constructs which are derived from alphaviruses. Briefly, within one aspect of the present invention alphavirus vector constructs are provided, comprising a 5′ promoter which initiates synthesis of viral RNA in vitro or in vivo from cDNA, a 5′ sequence which initiates transcription of alphavirus RNA, a nucleic acid molecule which operably encodes all four alphaviral nonstructural proteins including an isolated nucleic acid molecule as described above, an alphavirus RNA polymerase recognition sequence and a 3′ polyadenylate tract. Within other aspects, alphavirus RNA vector replicons are provided, comprising 5′ viral sequences required in cis for replication (also referred to as 5′ CSE, in background), sequences which, when expressed, code for biologically active alphavirus nonstructural proteins (e.g., nsP1, nsP2, nsP3, nsP4) including an nsP2 encoded by the isolated nucleic acid molecules described above, 3′ viral sequences required in cis for replication (also referred to as 3′ CSE, in background), and a polyadenylate tract. Each of these aspects is discussed in more detail below.

### 5′ Promoters Which Initiate Synthesis of Viral RNA

As noted above, within certain embodiments of the invention, alphavirus vector constructs are provided which contain 5′ promoters that can be used to initiate synthesis of alphaviral RNA from cDNA by in vitro or in vivo transcription. Within preferred embodiments such promoters for in vitro transcription include, for example, the bacteriophage T7, T3, and SP6 RNA polymerase promoters. Similarly, eukarytoic layered vector initiation systems are provided which contain 5′ promoters that can be used to initiate synthesis of viral RNA from cDNA in vivo (i.e., within a eukaryotic cell). Within certain embodiments, promoters for in vivo transcription are RNA polymerase II promoters and include, for example, viral simian virus 40 (SV40) (e.g., early or late), cytomegalovirus (CMV) (e.g., immediate early), Moloney murine leukemia virus (MOMLV) or Rous sarcoma virus (RSV) LTR, and herpes simplex virus (HSV) (thymidine kinase) promoters.

### Sequences Which Initiate Transcription

As noted above, within preferred embodiments the alphavirus vector constructs and RNA vector replicons of the present invention contain a 5′ sequence which is capable of initiating transcription of an alphavirus RNA (also referred to as 5′-end CSE, or 5′ cis replication sequence, see Strauss and Strauss, Microbiol. Rev. 58:491-562, 1994). Representative examples of such sequences include nucleotides 1-60, and to a lesser extent, nucleotides through bases 150-210, of the wild-type Sindbis virus, nucleotides 10-75 for tRNA^{Asp} (aspartic acid, Schlesinger et al., U.S. Pat. No. 5,091,309), and 5′ sequences from other alphaviruses which initiate transcription. It is the complement of these sequences, which corresponds to the 3′ end of the of the minus-strand genomic copy, which are bound by the nsP replicase complex, and possibly additional host cell factors, from which transcription of the positive-strand genomic RNA is initiated.

### Alphavirus Nonstructural Proteins

The alphavirus vector constructs and RNA vector replicons provided herein also require sequences encoding all four alphaviral nonstructural proteins, including a nsP2 sequence which provides the desired phenotype. Briefly, a wide variety of sequences that encode alphavirus nonstructural proteins (see Strauss and Strauss, Microbiol. Rev. 58:491-562, 1994), in addition to those explicitly provided herein, may be utilized in the present invention, and are therefore deemed to fall within the scope of the phrase “alphavirus nonstructural proteins.” For example, due to the degeneracy of the genetic code, more than one codon may code for a given amino acid. Therefore, a wide variety of nucleic acid sequences encoding alphavirus nonstructural proteins may be generated. Furthermore, amino acid substitutions, additions, or deletions at any of numerous positions may still provide functional or biologically active nonstructural proteins. Within the context of the present invention, alphavirus nonstructural proteins are deemed to be biologically active if they promote self-replication of the vector construct (i.e., replication of viral nucleic acids and not necessarily the production of infectious virus) and this replication may be readily determined by metabolic labeling or RNase protection assays performed over a time course. Methods for making such derivatives are readily accomplished by one of ordinary skill in the art given the disclosure provided herein.

Viral Junction Regions

The alphavirus viral junction region promoter normally controls transcription initiation of the subgenomic mRNA. Thus, this element is also referred to as the subgenomic mRNA promoter. In the case of Sindbis virus, the normal viral junction region typically begins at approximately nucleotide number 7579 and continues through at least nucleotide number 7612 (and possibly beyond). At a minimum, nucleotides 7579 to 7602 are believed necessary for transcription of the subgenomic fragment. This region (nucleotides 7579 to 7602) is hereinafter referred to as the “minimal junction region core.”

### Alphavirus RNA Polymerase Recognition Sequence, and Poly(A) Tract

As noted above, the alphavirus vectors should include an alphavirus RNA polymerase recognition sequence (also termed “alphavirus replicase recognition sequence”, “3′ terminal CSE”, or “3′ cis replication sequence”, see Strauss and Strauss, Microbiol. Rev. 58:491-562, 1994). Briefly, the alphavirus RNA polymerase recognition sequence, which is located at the 3′ end region of positive stranded genomic RNA, provides a recognition site at which replication begins with synthesis of the negative strand. A wide variety of sequences may be utilized as an alphavirus RNA polymerase recognition sequence. For example, within one embodiment, vector constructs in which the polymerase recognition is truncated to the smallest region that can still function as a recognition sequence (e.g., nucleotides 11,684 to 11,703 for Sindbis) can be utilized. Within another embodiment of the invention, vector constructs in which the entire nontranslated region downstream from the E1 gene to the 3′ end of the viral genome including the polymerase recognition site (e.g., nucleotides 11,382 to 11,703 for Sindbis), can be utilized.

Within preferred embodiments of the invention, the alphavirus vector construct or RNA vector replicon may additionally contain a poly(A) tract, which increases dramatically the observed level of heterologous gene expression in cells transfected with alphavirus-derived vectors (see e.g., Dubensky et al, supra). Briefly, the poly(A) tract may be of any size which is sufficient to promote stability in the cytoplasm and recognition by the replicase, thereby increasing the efficiency of initiating the viral life cycle. Within various embodiments of the invention, the poly(A) sequence comprises at least 10 adenosine nucleotides, and most preferably, at least 25 or 40 adenosine nucleotides. Within one embodiment, the poly(A) sequence is attached directly to Sindbis virus nucleotide 11,703.

### Eukaryotic Layered Vector Initiation Systems

Within one aspect of the present invention DNA-based vectors (referred to as “Eukaryotic Layered Vector Initiation Systems”) are provided that are capable of directing the synthesis of a self-replicating vector RNA in vivo. Generally, eukaryotic layered vector initiation systems comprise a 5′ promoter that is capable of initiating in vivo (i.e., within a cell) the 5′ synthesis of RNA from cDNA, a construct that is capable of directing its own replication in a cell, the construct also being capable of expressing a heterologous nucleic acid sequence, and a 3′ sequence that controls transcription termination (e.g., a polyadenylate tract). Such eukaryotic layered vector initiation systems provide a two-stage or “layered” mechanism that controls expression of heterologous nucleotide sequences and are described more comprehensively in U.S. Pat. No. 5,814,482 and U.S. Pat. No. 6,015,686. Representative 5′ promoters suitable for use within the present invention include RNA pol I, II, or III promoters, and may be inducible or non-inducible (i.e., constitutive) promoters, such as, for example, Moloney murine leukemia virus promoters, metallothionein promoters, the glucocorticoid promoter, Drosophila actin 5C distal promoter, SV40 promoter, heat shock protein 65 promoter, heat shock protein 70 promoter, immunoglobulin promoters, mouse polyoma virus promoter (Py), Rous sarcoma Virus (RSV), herpes simplex virus (HSV) promoter, BK virus and JC virus promoters, mouse mammary tumor virus (MMTV) promoter, CMV promoter, Adenovirus E1 or VA1 RNA promoters, rRNA promoters, tRNA methionine promoter, tetracycline responsive promoter, and the lac promoter.

The second layer comprises an autocatalytic vector construct which is capable of expressing one or more heterologous nucleotide sequences and of directing its own replication in a cell, either autonomously or in response to one or more factors (e.g. is inducible). The second layer may be of viral or non-viral origin. Within one embodiment of the invention, the second layer construct may be an alphavirus vector construct as described above. Replication competency of the autocatalytic vector construct, contained within the second layer of the eukaryotic vector initiation system, may be measured by a variety of assays known to those of skill in the art including, for example, ribonuclease protection assays which measure increases of both positive-sense and negative-sense RNA in transfected cells over time, in the presence of an inhibitor of cellular RNA synthesis, such as dactinomycin, and also assays which measure the synthesis of a subgenomic RNA or expression of a heterologous reporter gene in transfected cells.

Within particularly preferred embodiments of the invention, eukaryotic layered vector initiation systems are provided that comprise a 5′ promoter which is capable of initiating in vivo the synthesis of alphavirus RNA from cDNA (i.e., a DNA promoter of RNA synthesis), followed by a 5′ sequence which is capable of initiating transcription of an alphavirus RNA, a nucleic acid sequence which operably encodes all four alphaviral nonstructural proteins (including a nucleic acid molecule of the present invention that results in the desired phenotype), a subgenomic junction region promoter (modified or non-modified), a heterologous sequence to be expressed, an alphavirus RNA polymerase recognition sequence, and a 3′ sequence which controls transcription termination.

Heterologous Sequences

As noted above, a wide variety of nucleotide sequences may be carried and expressed by the vectors of the present invention, including, for example, sequences which encode palliatives such as lymphokines, cytokines, or chemokines (e.g., IL-2, IL-12, GM-CSF), prodrug converting enzymes (e.g., HSV-TK, VZV-TK), antigens which stimulate an immune response (e.g., from HIV, HCV), proteins for therapeutic application such as growth or regulatory factors (e.g., EPO, FGF, PDGF, VEGF), proteins which assist or inhibit an immune response, as well as ribozymes and antisense sequences (or sense sequences for “antisense applications”), and include those referenced previously (U.S. Pat. No. 6,015,686 and U.S. Pat. No. 6,015,694). The above described sequences may be obtained readily by one of skill in the art from repositories, cloned from cellular RNA using published sequences, or synthesized, for example, on an Applied Biosystems Inc. DNA synthesizer (e.g., APB DNA synthesizer model 392 (Foster City, Calif.)).

### Methods for Delivery of Vectors and Particles

As noted above, the present invention also provides methods for delivering a selected heterologous sequence to a vertebrate (e.g., a mammal such as a human or other warm-blooded animal such as a horse, cow, pig, sheep, dog, cat, rat or mouse) or insect, comprising the step of administering to a vertebrate or insect a vector or particle as described herein which is capable of expressing the selected heterologous sequence. Delivery may be by a variety of routes (e.g., intravenously, intramuscularly, intradermally, intraperitoneally, subcutaneously, orally, intraocularly, intranasally, intradermally, intratumorally, vaginally, rectally), or by various physical methods such as lipofection (Feigner et al., Proc. Natl. Acad. Sci. USA 84:7413-7417,1989), direct DNA injection (Fung et al., Proc. Natl. Acad. Scl. USA 80:353-357, 1983; Seeger et al., Proc. Natl. Acad. Sci. USA 81:5849-5852; Acsadi et al., Nature 352:815-818, 1991); microprojectile bombardment (Williams et al., PNAS 88:2726-2730, 1991); liposomes of several types (see, e.g., Wang et al., PNAS 84:7851-7855, 1987); CaPO₄ (Dubensky et al., PNAS 81:7529-7533, 1984); DNA ligand (Wu et al, J. Biol. Chem. 264:16985-16987, 1989); administration of nucleic acids alone (WO 90/11092); or administration of DNA linked to killed adenovirus (Curiel et al., Hum. Gene Ther. 3:147-154, 1992); via polycation compounds such as polylysine, utilizing receptor specific ligands; as well as with psoralen inactivated viruses such as Sendai or Adenovirus. In addition, the vectors and particles may either be administered directly (i.e., in vivo), or to cells which have been removed (ex vivo), and subsequently returned.

### Production of Recombinant Proteins

In another aspect of the present invention, alphavirus replicons, particles, vector constructs and eukaryotic layered vector initiation systems with the non-cytopathic phenotype described herein can be utilized to direct the expression of one or more recombinant proteins in eukaryotic cells (ex vivo, in vivo, or established cell lines). As used herein, a “recombinant protein” refers to a protein, polypeptide, enzyme, or fragment thereof. Using this approach, proteins having therapeutic or other commercial application can be more cost-effectively produced. Furthermore, proteins produced in eukaryotic cells may be more authentically modified post-translationally (e.g., glycosylated, sulfated, acetylated, etc.), as compared to proteins produced in prokaryotic cells. Within this aspect, the alphavirus vector or particle encoding the desired protein is transformed, transfected, transduced or otherwise introduced into a suitable eukaryotic cell. In certain instances an alphavirus replicon vector according to the present invention may be synthesized (e.g., transcribed) from DNA within the eukaryotic cell (see U.S. Pat. Nos. 6,015,686 and 5,814,482), through the use of an alphavirus vector construct or eukaryotic layered vector initiation system. Synthesis of the alphavirus replicon vector itself or gene expression from the vector may be inducible, by incorporating one or more additional elements (e.g., inducible RNA polymerase 11 promoter, temperature sensitive replicase genes, translationally regulated subgenomic mRNA).

Representative examples of proteins which can be produced using these approaches include, but are not limited to, insulin (see U.S. Pat. No. 4,431,740 and BE 885196A), hemoglobin (Lawn et al., Cell 21:647-51, 1980), erythropoietin (EPO; see U.S. Pat. No. 4,703,008), megakaryocyte growth and differentiation factor (MGDF), stem cell factor (SCF), G-CSF (Nagata et al., Nature 319:415-418, 1986), GM-CSF, M-CSF (see WO 8706954), the flt3 ligand (Lyman et al. (1993), Cell 75:1157-1167), EGF, acidic and basic FGF, PDGF, members of the interleukin or interferon families, supra, neurotropic factors (e.g., BDNF; Rosenthal et al., Endocrinology 129:1289-1294, 1991, NT-3; see WO 9103569, CNTF; see WO 9104316, NGF; see WO 9310150), coagulation factors (e.g., factors VIII and IX), thrombolytic factors such as t-PA (see EP 292009, AU 8653302 and EP 174835) and streptokinase (see EP 407942), human growth hormone (see JP 94030582 and U.S. Pat. No. 4,745,069) and other animal somatotropins, integrins and other cell adhesion molecules, such as ICAM-1 and ELAM (see also other “heterologous sequences” discussed above), and other growth factors, such as VEGF, IGF-I and IGF-II, TGF-β, osteogenic protein-1 (Ozkaynak et al., EMBO J. 9:2085-2093, 1990), and other bone or cartilage morphogenetic proteins (e.g., BMP-4, Nakase et al, J. Bone Miner. Res. 9:651-659, 1994). As those in the art will appreciate, once characterized, any gene can be readily cloned into vectors of the present invention, followed by introduction into a suitable host cell and expression of the desired gene. In addition, such vectors may be delivered directly in vivo, either locally or systemically to promote the desired therapeutic effect (e.g., wound healing applications). A variety of eukaryotic host cell lines (e.g., COS, BHK, CHO, 293, or HeLa cells) may be used to produce the desired protein.

The following examples are included to more fully illustrate the present invention. Additionally, these examples provide preferred embodiments of the invention and are not meant to limit the scope thereof. Standard methods for many of the procedures described in the following examples, or suitable alternative procedures, are provided in widely reorganized manuals of molecular biology, such as, for example, “Molecular Cloning,” Second Edition (Sambrook et al., Cold Spring Harbor Laboratory Press, 1987) and “Current Protocols in Molecular Biology” (Ausubel et al., eds. Greene Associates/Niley Interscience, NY, 1990).

### EXAMPLES

### Example 1

### Isolation and Characterization of Noncytopathic Sin and SFV Replicons

The following example describes the identification and molecular characterization of alphavirus replicon variants that exhibit reduced inhibition of host macromolecular synthesis and are capable of establishing persistent infection in vertebrate cells, as compared to their cytopathic parental “wild-type” strains. Briefly, to select non-cytopathic alphavirus replicon variants, the neomycin phosphotransferase gene (neo) was placed under the control of the subgenomic promoter in Sindbis virus (SIN) and Semliki Forest virus (SFV) derived replicons to generate the constructs pSINBV-neo and pSFV-neo as follows. The neomycin phosphotransferase gene was isolated by standard PCR amplification (10 cycles of 30 sec at 94° C., 30 sec at 55° C., 2 min at 72° C.) from plasmid pcDNA3 (Invitrogen, San Diego, Calif.) using primers designed to flank the gene with either XhoI and NotI (for pSINBV-neo) or BamHI (for pSFV-neo) restriction sites:

| Replicon | Forward primer | Reverse primer |
|---|---|---|
| SIN | NeoFX | NeoRN |
| | 5′ ATATACTCGAGACCATGA | 5′ TATATAGCGGCCGCTCAG |
| | TTGAACAAGATGGATTG-3′ | AAGAACTCGTCAAGAAG-3′ |
| | (SEQ ID NO: 1) | (SEQ ID NO: 2) |
| SFV | 5′ BAMHI-Neo | 3′ BAMHI-Neo |
| | 5′ ATATAGGATCCTTCGCAT | 5′ ATATAGGATCCTCAGAAG |
| | GATTGAACAAGATGGATTGC- | AACTCGTCAAGAAGGCGA-3′ |
| | 3′ | (SEQ ID NO: 4) |
| | (SEQ ID NO: 3) | |

Following amplification, the DNA fragments were purified with QIAquick-spin (Qiagen) and digested with XhoI and NotI, or BamHI. The neo resistance gene flanked by XhoI and NotI was ligated into pRSIN-βgal (Dubensky et al., “Sindbis Virus DNA-based Expression Vectors: Utility For In Vitro and In Vivo Gene Transfer,” J. Virol. 70:508-519 (1996)) vector that had been digested with XhoI and NotI, treated with calf intestinal alkaline phosphatase, and purified away from its previous βgalactosidase insert, using a 0.7% agarose gel and QIAEX II (Qiagen), generating pSNBV-Neo. The neo gene flanked by BamHI was ligated into pSFV-1 vector that had been digested with BamHI, treated with calf intestinal alkaline phosphatase, and purified from a 0.7% agarose gel, generating pSFV-Neo. These plasmid constructs were linearized (pSINBV-neo with PmeI, pSFV-neo with SpeI) and in vitro transcribed with SP6 polymerase (Promega) in the presence of CAP analog (New England Biolabs). In some selection experiments, the RNA was transcribed from linear DNA that had previously been subjected to 1, 2, 3, or 4 rounds of mutagenesis by passage through E. coli strain XL-1 Red (Stratagene). Replicon RNAs were transfected into BHK cells and, 24 hrs later, the cells were subjected to G418 (Geneticin, GIBCO BRL, 0.5 mg/ml) selection. Approximately 24 hour post-transfection, the BHK cells were trypsin treated and plated in medium containing 0.5 mg/ml G418. Subsequently, the medium was changed at approximately 24 hour intervals to remove dead cells, and replaced with G418-containing medium. Using this selection, all cells in control plates transfected with replicon expressing βgal were killed by the drug. Stable neomycin resistant colonies were obtained for both mutagenized and non-mutagenized SIN-neo and SFV-neo replicons. In addition, neo resistant colonies were obtained after infection of BHK cells with packaged vector particles containing non-mutagenized replicon generated as previously described. (Polo et al., “Stable alphavirus packaging cell lines for Sindbis virus and Semliki Forest virus-derived vectors.” Proc Natl Acad Sci USA. 96:4598603 (1999)). These data indicated that the phenotype was associated with replicon RNA rather than contaminating plasmid DNA.

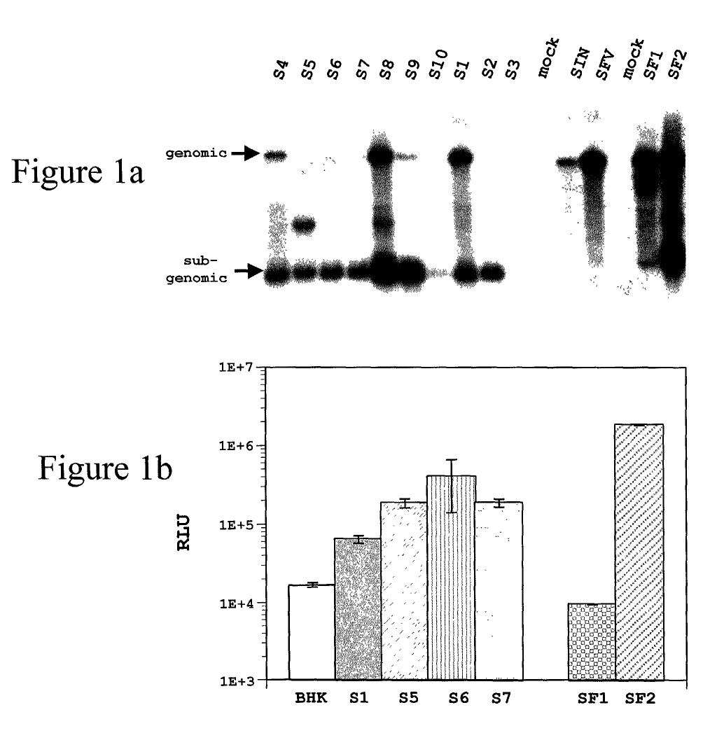

Within each selection, the drug-resistant BHK cells were pooled and expanded. To confirm that neo expression was associated with RNA species corresponding to alphavirus replication, polyA-mRNA was extracted from the pools (Triazol, BRL, followed by Oligotex, Qiagen) and analyzed by Northern blot hybridization with a ³²P-labeled DNA fragment derived from the neo resistance gene (FIG. 1A). The SIN-derived pools were designated S1-S10 and the SFV derived pools were designated SF1-2. The polyA-selected RNA was extracted from BHK cells either transfected (lanes S1-2, S4-10, and SF1-2) or infected (lane S3) with vector RNAs and selected with G418. Pools were obtained from non-mutagenized replicon (lanes S1-3 and SF1), from replicons transcribed from templates that had been subjected to one round (lanes S4, S7), two rounds (lanes S8, SF2), three rounds (lanes S5, S9), and four rounds (S6, S10) of mutagenesis. In vitro transcribed genomic RNA from the two vectors was loaded as markers in lanes SIN and SFV and polyA-selected mRNA from naive BHK cells was loaded in lanes E. The expected sizes for genomic SIN replicon is 8.8 kb and for SFV is 9 kb, while the expected sizes for vector subgenomic RNA are 1.2 kb for SIN and 1.65 kb for SFV. FIG. 1A shows that the neo sequence was found within both genomic and subgenomic length RNA species for all pools. Furthermore, this analysis indicated that the RNA profiles varied significantly among the SIN and SFV pools, particularly with respect to the relative ratios between subgenomic and genomic RNA, and the appearance of new RNA species migrating faster than the genomic RNA (lanes S5, S8, S9, SF1, and SF2). These data suggested possible phenotypic differences among the selected variants.

To further confirm that neo resistance was conferred by the replicon, naive BHK cells were electroporated with 5-10 μg of polyA-mRNA extracted from either SIN- or SFV-derived neo resistant pools or from other naive BHK cells as control. Approximately 48 hrs later, the transfected cells were subjected to G418 selection. Transfection with mRNA from both SIN and SFV derived pools rapidly generated such high numbers of neo resistant cells that individual drug resistant colonies could not be counted. In contrast, control mRNA gave no colonies over an extended period of time.

To determine whether vector RNA was actively replicating in neo resistant cells, stable drug-selected pools were transfected with defective replicons encoding a βgal reporter gene, but deleted of the nonstructural genes. Amplification and subgenomic transcription of the βgal mRNA in these vectors could occur only if the nsPs are provided in trans by the replicon already present in the neo resistant pools. The defective replicon RNAs were transcribed from plasmids pSINBVdInsP-βgal (derived from pSINBV-βgal [Dubensky, 1996 #15] by deleting the BspEI fragments), and pSFV3dInsP-βgal (derived from pSFV3-βgal (Liljestrom et al., “in vitro Mutagenesis of a Full-Length cDNA Clone of Semliki Forest Virus: The Small 6,000-molecular Weight Membrane Protein Modulates Virus Release,” J. Virol. 65:4107-4113 (1991)), GIBCO-BRL, by deleting the PstI fragments). After introduction of the defective βgal replicons into SIN- and SFV-derived neo resistant pools, βgal expression was measured using the Luminescent β-galactosidase assay kit (Clontech). FIG. 1B shows the results of this complementation analysis. βgal detection was measured in relative light units and in all but one pool βgal was detected. This result clearly demonstrated that the variant replicons were actively replicating in cells in order to provide trans-complementation. Pool SF1 did not show demonstrable βgal expression, indicating a defect reducing either the replication or the subgenomic transcription in trans.

### Example 2

### The Genetic Determinants Associated With a Non-Cytopathic Phenotype

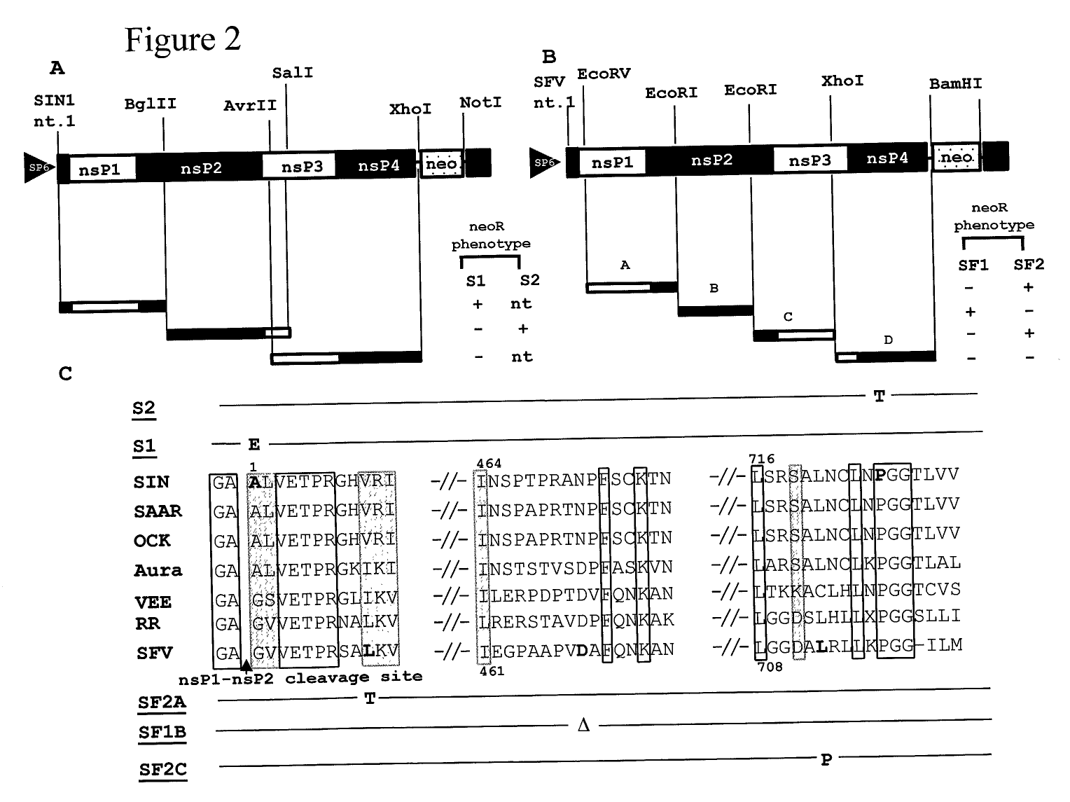

To identify the mutations responsible for a non-cytopathic phenotype, representative pools (S1, S2, Sf1, and SF2) were chosen based on the different RNA profiles in the Northern analysis. All nsP genes of the SIN and SFV variant replicons present in these pools were cloned by RT-PCR in three or four fragments, respectively (FIG. 2A and B), with the following primer sets:

| | Fragment | Negative-sense | Primer pairs for PCR |
|---|---|---|---|
| Replicon | Coordinates | primers | amplification |
| SIN | 1-2288 | nsP2R | SINSP6F |
| | Apa I-Bgl II | 5′ ATTATAAGCTT | 5′ TATATGGGCCCGATTTAGG |
| | | GGCTCCAACTCCAT | TGACACTATAGATTGACGGCGT |
| | | CTC-3′ | AGTACAC-3′ |
| | | (SEQ ID NO: 5) | (SEQ ID NO: 6) |
| | | | SIN2355R |
| | | | 5′ TATATGGATCCCTCAGTCT |
| | | | TAGCACGTCGGCCTC-3′ |
| | | | (SEQ ID NO: 7) |
| SIN | 2288-4845 | nsP3R | nsP2F |
| | Bgl II-Sal | 5′ ATATATCTCGA | 5′ ATTATGGATCCGGCATTAG |
| | | GGTATTCAGTCCTC | TTGAAACCCCG-3′ |
| | | CTGCT-3′ | (SEQ ID NO: 9) |
| | | (SEQ ID NO: 8) | |
| | | | SIN4897R |
| | | | 5′ TATATGGTACCATGCAAAG |
| | | | GCACGGCAACGTTTTG |
| | | | (SEQ ID NO: 10) |
| SIN | 4281-7645 | SIN11703R | nsP3F |
| | Avr II-Xho I | 5′ GAAATGTTAAA | 5′ TATATGAATTCGCGCCGTC |
| | | AACAAAATTTTGTT | ATACCGCACC |
| | | GA-3′ | (SEQ ID NO: 12) |
| | | (SEQ ID NO: 11) | |
| | | | SFneoBHI/F |
| | | | 5′ ATATACGGAGAACCTGCGT |
| | | | GCAATCCATC-3′ |
| | | | (SEQ ID NO: 13) |
| SFV | 162-2184 | SF2158R | SFV162F |
| | EcoR V-EcoR I | 5′ ATATACTACTA | 5′ ATATAGGAGACTGACAAAG |
| | | CTGTAGTCTTATAT | ACACACTCA-3′ |
| | | GGTG-3′ | (SEQ ID NO: 15) |
| | | (SEQ ID NO: 14) | |
| | | | SFV2129R |
| | | | 5′ ATATAGGCCTGATCTTCAG |
| | | | CCCTTCGTAG-3′ |
| | | | (SEQ ID NO: 16) |
| SFV | 2184-3762 | SF3668R | SFV2184F |
| | EcoR I-EcoR I | 5′ ATATACCAAGC | 5′ ATATAGTTGGTGGGAGAGC |
| | | ATCTGCAGCTCATG | TAACCAACC-3′ |
| | | GCG-3′ | (SEQ ID NO: 18) |
| | | (SEQ ID NO: 17) | |
| | | | SFV3709R |
| | | | 5′ ATATACGACACACTGCTGG |
| | | | TAGTGGTGG-3′ |
| | | | (SEQ ID NO: 19) |
| SFV | 3762-5304 | SFV5255R | SFV3640F |
| | EcoR I-Xho I | 5′ ATATAGCTCTC | 5′ ATATAGGCAGGTTCGACTT |
| | | TTCGGGCGCGGTGG | GGTCTTTGTG-3′ |
| | | AG-3′ | |
| | | (SEQ ID NO: 20) | SFV5255R |
| | | | (SEQ ID NO: 21) |
| SFV | 5305-7400 | SFneoBHI/F | SFV5185F |
| | Xho I-BamH I | 5′ ATATACGGAGA | 5′ ATATAGATGTGCACCCTGA |
| | | ACCTGCGTGCAATC | ACCCCGCAGAC-3′ |
| | | CATC-3 | |
| | | (SEQ ID NO: 22) | SFneoBHI/F |
| | | | (SEQ ID NO: 23) |

The cDNAs were synthesized using polyA-mRNA extracted from the neo resistant pools as templates, the Superscript Pre-amplification kit (GIBCO-BRL) and negative sense primers as indicated in above table. These cDNAs were amplified by 25 PCR cycles with either Vent Polymerase (NEB) or Pfu (Stratagene) with primer pairs either overlapping or adjacent to each restriction site (see above table). Amplified fragments then were used to replace the corresponding fragment in wild-type pSINBV-neo or pSFV-neo using the restriction sites indicated in the table. Replicon RNA transcribed in vitro from three independent clones for each substituted fragment was transfected into naive BHK cells. Following G418 selection, the number of colonies obtained for each construct was compared to the number of colonies obtained with the parental wild-type replicon. FIGS. 2A and 2B show schematics of the cloning strategy used to map the vector variants. FIG. 2A shows the diagram of the SINBV-neo construct and FIG. 2B shows the diagram of the SFV-neo construct. The fragments that were amplified by RT-PCR using polyA-selected RNA from the pools are shown with nsP coding sequences. Restriction sites used in the cloning are also indicated. The ability of each fragment substitution to generate high numbers of neo resistant BHK cells (+) as compared to the parental vectors (−) is shown. Some fragments were not tested (indicated as nt). As summarized in the Figure, a single specific fragment was found to provide the neo resistant phenotype in most pools. For the SF2 pool, which was derived from vector that had undergone two rounds of mutagenesis, two fragments independently conferred the phenotype. Thus both SIN and SFV replicons that established persistent replication were generated with a defined fragment.

The defined fragments were sequenced entirely and compared to the parental replicon sequence. In FIG. 2C, the sequence alignment of the nsP2 regions in which the mutations were located is shown for several alphaviruses. Bold characters indicate amino acid residues where mutations were found and the changes are indicated above the alignment for the SIN-derived variants and below the alignment for the SFV-derived variants. In variant SF1B, Δ indicates the deletion of the amino acid D₄₆₉. Since the length of nsP2 varies between SIN and SFV, codon numbering is indicated for both. White boxes highlight identical residues among all the alphaviruses aligned. Gray boxes highlight conservative changes. Interestingly, each SIN and SFV cloned variant contained only a single amino acid substitution within the nsP2 protein. Although the precise location of these amino acid changes differed among the SIN and SFV variants, the amino terminus (aa1 in variant S1 and aa10 in variant SF2A) and a small region of the carboxy-terminus (aa726 in variant S2 and aa713 in variant SF2C) seemed to be targeted preferentially. The latter region is within the putative protease domain of nsP2 [Hardy, 1989 #29]. Interestingly, the S1 mutation mapped at the nsP1-nsP2 cleavage site [Strauss, 1994 #4], and the SF1B variant contained an in-frame deletion of amino acid 469 within yet another nsP2 region.

### Example 3

### Properties of the Cloned Non-Cytopathic Alphavirus Vector Variants

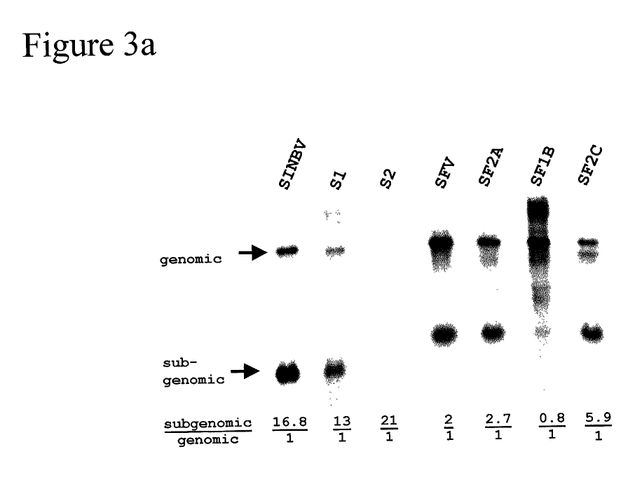

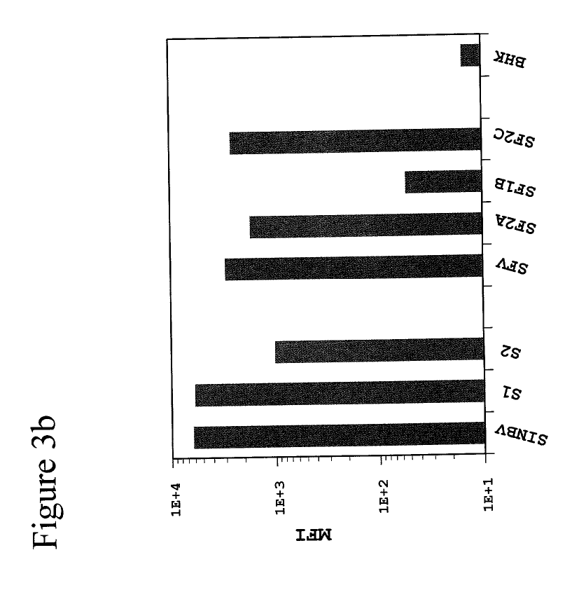

To characterize these cloned variants, the impact of each mutation on ratios of subgenomic and genomic RNA was examined. Drug-resistant cell lines obtained with the cloned SIN and SFV replicon variants and naive BHK cells electroporated 2 hrs earlier with parental replicon RNAs, were labeled with ³H uridine (Dryga et al., “Identification of mutations in a Sindbis virus variant able to establish persistent Infection in BHK cells: the importance of mutation in the nsP2. gene,” J. Virol. 228:74-83 (1997)). Total RNA was separated by gel electrophoresis (Sambrook et al., “Molecular cloning: A Laboratory Manual,” (2nd ed.) Cold Spring Harbor, Cold Spring Harbor, N.Y.(1989)), the gels were treated and exposed to film (Frolov et al., “Selection of RNA Replicons Capable of Persistent Noncytopathic Replication In Mammalian Cells,” J. Virol. 73:3854-3865 (1999)), and regions containing the genomic or subgenomic RNAs were excised and subjected to scintillation counting. FIG. 3A shows the results of this analysis with the cloned variant vectors in lanes S1, S2, SF2A, SF1B, and SF2C, and BHK cells electroporated two hours earlier with parental vector RNAs in lanes SINBV and SFV. Below the gel, the results of the scintillation counting are expressed as molar ratio of subgenomic to genomic RNA. Although a direct comparison could not be made with the transiently transfected parental vectors, the variant replicons clearly showed different molar ratios of subgenomic to genomic RNA when compared to each other. This result suggested that the nsP2 mutations affected the levels of genomic replication and/or subgenomic transcription. Also, it appeared that some variants, S2 and SF2C, had reduced amounts of genomic RNA when compared to other variants (same number of cells were labeled and similar amounts of total RNA were loaded on the gel).

To examine the effect of these mutations on subgenomic transcription, the expression levels of an E-GFP reporter gene (Clontech) was compared between variant and parental replicons. BHK cells were electroporated with in vitro transcribed replicon RNA and assayed 24 hrs later for GFP expression by flow cytometry and the mean fluorescence intensity (MFI) of the GFP positive cell population was plotted (FIG. 3B). Data are the average from two independent electroporations done on the same day and are representative of several similar experiments. Although the efficiency of transfection varied among replicons, the GFP expression within individual transfected cells clearly was equivalent to the parental replicons for all but SF1B. Since the ratio of subgenomic to genomic RNA in SF1B was lower than in the other variants (FIG. 3A), deletion of D₄₆₉ might affect the subgenomic transcription.

Whether the mutations differentially affected plus strand or minus strand RNA synthesis was also analyzed. To differentiate the levels of each RNA species, semiquantitative RT-PCR was performed on equivalent amounts of total RNA extracted from either neo resistant BHK cell lines containing the cloned SIN and SFV variant replicons or naive BHK cells electroporated 24 hrs earlier with the parental replicons. Oligonucleotides complementary to either plus or minus strand RNA were used for detection of plus or minus strand cDNA respectively as indicated below.

| | Primer for | Primer for | Primer pairs |
|---|---|---|---|
| | detection of | detection of | for PCR |
| Replicon | minus strand | plus strand | amplification |
| SIN | SIN4795F | SIN6984R | SING161F |
| | 5′ TATTACCCGG | 5′ TATTACCCGG | 5′ CTATCCGACAGTAGCA |
| | GTGCCTACATATT | GTGCGCACTCGAT | TCTTATCAG-3′ |
| | GGGTGAGACCATG | CAAGTCGAGTAGT | (SEQ ID NO: 26) |
| | -3′ | G-3′ | |
| | (SEQ ID NO: 24) | (SEQ ID NO: 25) | SIN6860R |
| | | | 5′ GTCGCCTGCTTGAAGT |
| | | | GTTCTG-3′ |
| | | | (SEQ ID NO: 27) |
| SFV | SFV3640F | SFV5255R | SFV4551F |
| | see table 1 | see table 1 | 5′ GAAGCCATTGACATGA |
| | | | GGACGGC-3′ |
| | | | (SEQ ID NO: 28) |
| | | | SFV5250R |
| | | | 5′ CTGCGGGTTCAGGGTG |
| | | | TACGTC-3 |
| | | | (SEQ ID NO: 29) |

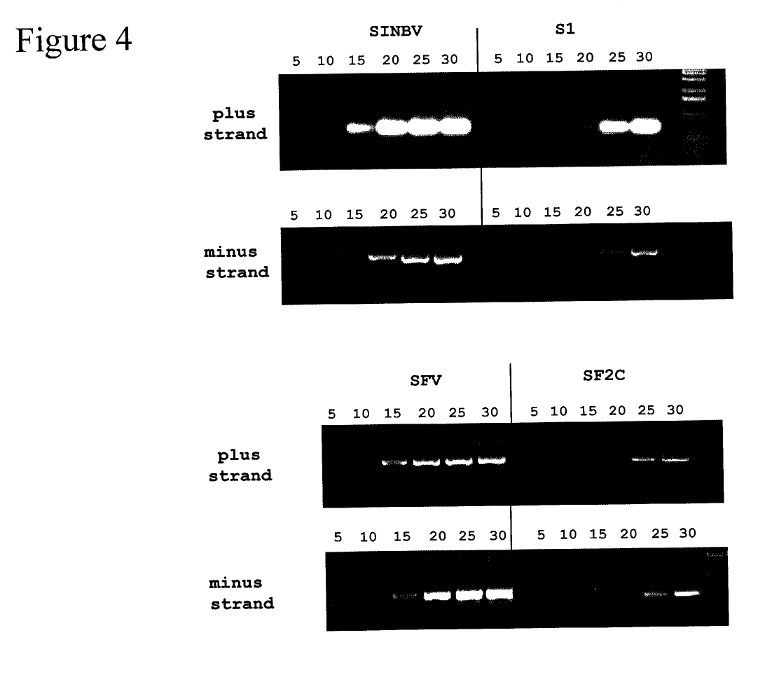

After cDNA synthesis and RNase A treatment, a 700 bp fragment corresponding to a region of either nsP4 for SIN variants or nsP3 for SFV variants was amplified by PCR using the appropriate primer pairs as indicated in the table above. Each PCR reaction was divided into several aliquots. Every 5 amplification cycles, one aliquot was removed and frozen for subsequent gel electrophoresis analysis. FIG. 4 shows the detection of minus strand and plus strand RNA by RT-PCR for variants S1 and SF2C, and parental replicons (SINBV and SFV). The PCR amplification cycle in which each aliquot was removed is indicated above each lane. Both plus and minus strand RNA levels were similarly lower with both S1 and SF2C variants as compared to the parental vectors at a 24 hr post-electroporation electroporation time point. Similar results were obtained with the other variants (data not shown). The cDNA for the housekeeping gene BHKp23 (Rojo et al., “Involvement of the Transmembrane Protein p23 in Biosynthetic Protein Transport,” J. Cell Biol. 139:1119-1135(1997)) also was synthesized from each sample as an internal standard. Oligo-dT was used to prime the reverse transcription and the following primer pair was used for the PCR amplification of a 700 bp fragment within the p23 gene.

| | |
|---|---|
| p23F | |
| 5′ATGTCTGGTTCGTCTGGCCCAC-3′, | (SEQ ID NO: 30) |
| p23R | |
| 5′CTCTATCAACTTCTTGGCCTTGAAG-3′ | (SEQ ID NO: 31) |

This PCR amplification reaction also was divided into several aliquots. Every 5 amplification cycles, one aliquot was removed and frozen for subsequent gel electrophoresis analysis. Similar amounts of product were obtained in all cases (data not shown). This result clearly demonstrated that each variant has ongoing minus strand synthesis, which is a requirement for persistent replication.

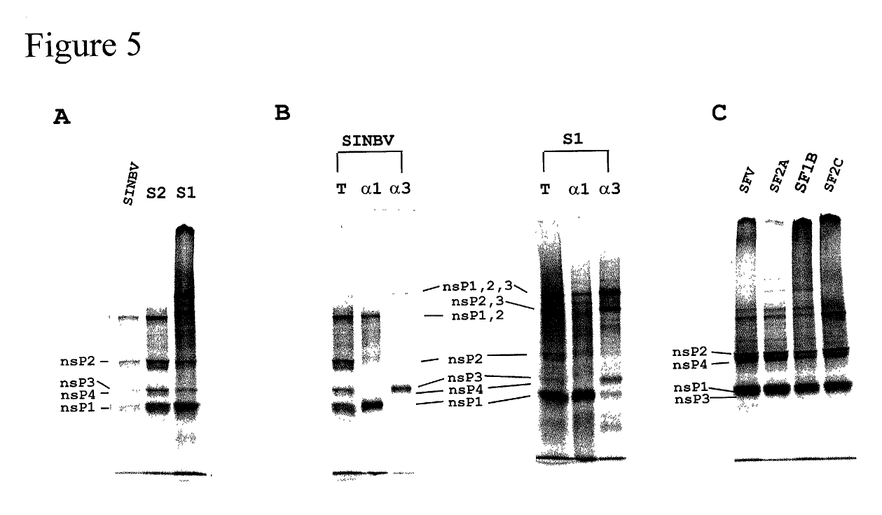

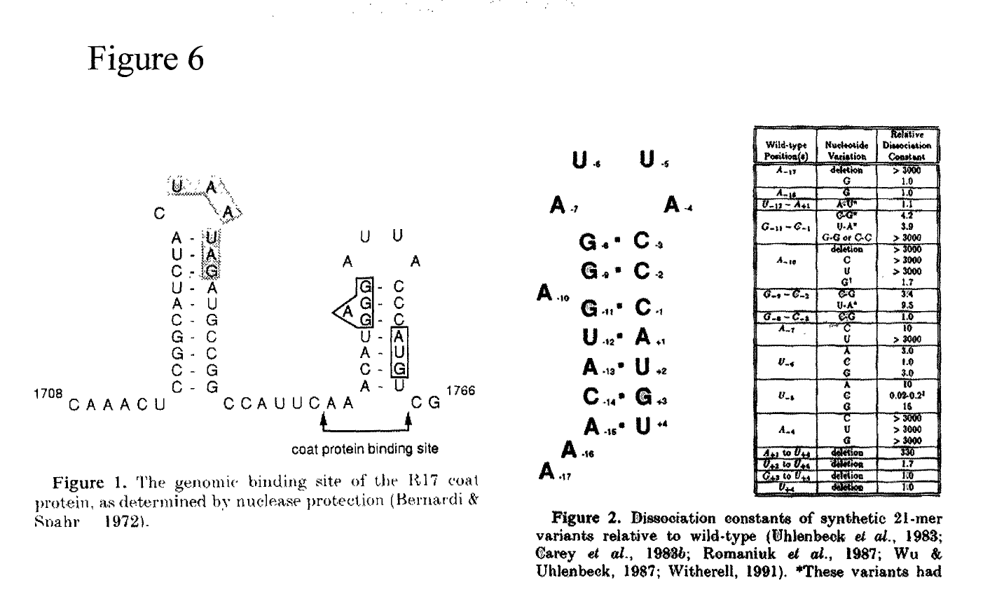

Alphavirus nsPs are translated initially as two polyproteins, P1234 and P123+P4. These polyproteins are processed subsequently into mature monomers by the nsP2 protease (Ding et al., “Evidence that Sindbis Virus NSP2 is an Autoprotease Which Processes the Virus Nonstructural Polyprotein.” Virology 171:280-4, and Hardy et al., “Processing the Nonstructural Polyproteins of Sindbis Virus: Nonstructural Proteinase is in the C-terminal Half of nsP2 and Functions Both in cis and in trans.” J. Virol. 63:4653-64 (1989)), with the processing intermediates playing an important role in the early events of RNA replication including a shift from minus strand to plus strand synthesis (Strauss et al., “The Alphaviruses: Gene Expression, Replication, and Evolution.” 58:3491-562, and Sawicki et al., “Role of the Non-Structural Polyproteins in Alphaviral RNA Synthesis,” pp. 187-198. In Enjuanes (ed.), Coronaviruses and Anterviruses, Plenum Press, New York (1998)). Since minus strand synthesis was maintained with the SIN and SFV variant replicons, the effect of mutations on polyprotein processing was analyzed. Coupled transcription and translation of parental and variant replicon RNA was performed with rabbit reticulocyte lysates (TNT, Promega) in the presence of [³⁵S]-methionine (Amersham). The 8% SDS-PAGE analysis is shown in FIG. 5A for the SIN variant and parental replicons, and in FIG. 5C for SFV variant and parental replicons. This analysis revealed that although all mutants accumulated the nsP monomers, mutants S1, SF2A, and SF1 B also accumulated significant amounts of higher molecular weight products. Immunoprecipitation of the in vitro translated products from SINBV and S1 with antisera specific for either nsP1 or nsP3 was performed as follow. From the translation reaction, 85 μl was removed and diluted to 200 μl to have a final concentration of 150 mMNaCl, 20 mM Tris pH 8, 1 mMEDAT, 0.1% NP40 (IP buffer) and 25% ProteinA-sepharose (Pharmacia). The mixtures were incubated at 4° C. with gentle rocking for 1 hr. After a brief spin (15 sec) 30 μl aliquots of the supernatant were transferred into new tubes containing the antiserum specific either for nsPl or nsP3 which had been premixed 15 min earlier with 25 μl of 50% Protein A-Sepharose. As control, an aliquot of 30 μl was transferred into a tube containing only the Protein A-Sepharose. The mixtures were incubated at 4° C. for two hours with gentle rocking. After a brief spin, the Sepharose was washed 3 times with IP buffer and resuspended in protein sample buffer. FIG. 5B shows the analysis by 8%SDS-PAGE of the reactions immunoprecipitated with antiserum specific for either SIN nsP1 (lanes α1) or SIN nsP3 (lanes α3) and the untreated aliquot of the translation reaction (lane T). No background was observed in the reactions with only Protein-A Sepharose (data not shown). This analysis indicated that variant S1 accumulated the P123 and P23 precursors and suggested that the maintenance of minus strand synthesis maybe achieved through altered polyprotein processing.

Finally, the ability to package the variant replicons into virion-like particles was analyzed by supplementing the structural proteins in trans, from in vitro transcribed defective helper RNAs prepared as previously described [Polo, 1999 #38 ].

| | | |
|---|---|---|
| SINBV-GFP | 5 e8 | PFU/ml |
| S1-GEP | 3.8 e8 | PFU/ml |
| S2-GEP | ≦1 e4 | PFU/ml |
| SFV3-LacZ | 3.8 e8 | PFU/ml |
| SF2A-βgal | 5 e7 | PFU/ml |
| SF2C-βgal | 1 e7 | PFU/ml |
| SF1B-βgal | ≦1 e4 | PFU/ml |

Interestingly, and in contrast to all previously published observations, particular non-cytopathic variant replicons of the present invention, could be packaged as efficiently as the parental replicon (SINBV-GFP 5e8 PFU/ml vs. S1-GFP 3.8e8 PFU/ml), while others packaged with only a slightly decreased efficiency (SFV3LacZ 3.8e8 PFU/ml vs. SF2ALacZ 5e7 PFU/ml and SF2C₁e7 PFU/ml). This observation greatly expands the utility of such alphavirus derived vectors. The remaining replicons were packaged at very low efficiency (≦1e4 PFU/ml).

The variant replicons describe above also can be utilized in a DNA based configuration known as eukaryotic layered vector initiation systems (ELVIS, see U.S. Pat. Nos. 5,814,482 and 6,015,686). Modification of the above replicons into that configuration are readily accomplished by one of skill in the art using the teachings provided herein, as well as the referenced U.S. Patents. For example, the nonstructural protein 2 genes containing the S1 or S2 mutations were substituted into a DNA based SIN replicon vector further comprising the puromycin selectable marker. Plasmid pSINCPpuro was first constructed by obtaining the puromycin resistance marker from pPUR (Clontech) by digestion with ApaI, blunt-ending, and further digestion with PvuII. The puromycin fragment then was ligated into the SIN plasmid replicon vector pSINCP that had been digested with Psil to generate the construct pSINCPpuro. Insertion of the variant S1 and S2 sequences was by substitution of the BbvC1 to AfIII restriction fragment. The new constructs may be used directly or further modified (see below) for stable transformation into a desired cell line and selection using the puromycin drug.

### Example 4

### Recombinant Protein Expression

Alphavirus vectors as described herein may be used for expression of recombinant protein(s). One method of recombinant protein expression utilizes eukaryotic cells (e.g., mammalian, insect) which are stably transformed with an alphavirus vector construct or Eukaryotic Layered Vector Initiation System (see U.S. Pat. Nos. 5,814,482 and 6,015,686, incorporated by reference), containing an altered nsP2 gene of the present invention. Although such a method is useful for many recombinant proteins, this approach has less applicability for recombinant proteins that are toxic to the host cell. Similar to other expression systems, it is often difficult to generate stably transformed cell lines that constitutively express high levels of a toxic protein. In such instances, further modification to provide inducible control of the alphavirus vectors may be used to overcome these issues. Herein, compositions and methods are described for recombinant protein expression utilizing inducible eukaryotic layered vector initiation systems.

Specifically, in one example, stably transformed cell lines are generated, wherein expression of a heterologous protein from the alphavirus replicon is regulated inducibly in a temperature sensitive manner. In preferred embodiments, this strategy uses a ligand binding sequence, such as a translational operator sequence, incorporated into the replicon vector (e.g., 3′-end, 5′-end, subgenomic mRNA) and a temperature sensitive ligand, such as an RNA binding protein, supplied in trans, which specifically interacts with the ligand binding sequence, blocking RNA synthesis by the alphaviral replicase or translation by the ribosome complex.

For example, in one such embodiment, one or more copies of a translation operator (TOP) sequence may be inserted into the alphaviral 3′-end nontranslated region (NTR), upstream of the terminal conserved 19 nucleotides. At the permissive temperature, interaction with the appropriate temperature sensitive binding protein would occur, and thus prevent recognition of the replicon 3′-end and synthesis of minus strand RNA. Upon shifting to the non-permissive temperature, RNA binding no longer occurs and replicon amplification and heterologous gene expression is permitted to occur in an unobstructed manner, and thus is “induced”. Alternatively, subgenomic mRNA translation may be regulated as a temperature sensitive induction system by incorporating the TOP sequence(s) immediately after the subgenomic promoter and upstream of the heterologous gene to be expressed. Again, at the permissive temperature, interaction with the appropriate binding protein would occur, and thus prevent translation of the heterologous gene by the host cell ribosome complex. Upon shifting to the non-permissive temperature, RNA binding no longer occurs and translation of the heterologous protein is induced.

In one embodiment, the inducible regulatory elements comprise a temperature sensitive (ts) bacteriophage R17/MS2 coat protein and its associated translational operator. (TOP) binding site sequence (FIG. 6). As a first step, a previously undescribed ts R17/MS2 coat protein is derived by mutagenesis of an R17/MS2 expression cassette, and selection for the desired ts phenotype. The R17/MS2 coat protein gene is amplified from template plasmid (e.g., Peabody and Lim, Nucleic Acid. Res. 24:2352-2359, 1996) or template bacteriophage DNA (e.g., ATCC 15597-B1) using the following primers that contain flanking BamHI and HindIII sites:

MS2COATfwd:

(SEQ ID NO: 32)

5′-ATATATGGATCCATGGCTTCTAACTTTACTCAGTT
MS2COATrev:

(SEQ ID NO: 33)

5′-ATATATAAGCTTTTAGTAGATGCCGGAGUTGCTG

Following PCR amplification the R17/MS2 coat protein gene is purified using QIAquick, digested with BamHI and HindIII, and ligated into plasmid pCMV-Script (Stratagene, San Diego, Calif.) that has also been digested with BamHI and HindIII and purified from an agarose gel. This construct is designated pCMV-coat. Random mutagenesis of the coat protein gene is performed by growing the pCMV-coat plasmid in XL-1 Red Mutator strain of E coli (Stratagene). A preparation of mutated plasmid is isolated and used for transfection as outlined below.

For screening, a GFP reporter cell line is constructed that expresses a destabilized form of the GFP reporter, derived from plasmid pd2EGFP-N1 (Clontech, Palo Alto, Calif.), and which is modified to contain the R17/MS2 operator sequence in the 5′-end non-translated region preceding the ATG initiation codon. Similar cassettes may also be constructed to contain multiple R17/MS2 operators. The modified GFP cassette with operator(s) is constructed by PCR synthesis using plasmid pd2EGFP-N1 as template and the following primers that contain the operator sequence and flanking NheI and XbaI restriction sites:

R17GFPfwd:

(SEQ ID NO: 34)

5′-ATATATGCTAGCCATGAGGATCACCCATGGTCGCCACCATGGTGAGC

AAGGGC

R17GFPrev:

(SEQ ID NO: 35)

5′-ATATATTCTAGAGTCGCGGCCGCGCATCTAC

Following amplification, the PCR fragment is purified using QIAquick, digested with NheI and XbaI, and ligated into plasmid pd2EGFP-N1 that has also been digested with NheI and XbaI. The resulting construct, designated pR17GFP, is transfected into a mammalian cell line (e.g., BHK, 293) and at 24 hr post-transfection, the cells are subjected to drug selection using G418 (GIBCO/BRL, Rockville, Md.). Drug resistant colonies are subjected to dilution cloning and one or more GFP expressing cell lines are chosen for further use.

To identify candidate ts coat protein variants, pools of mutagenized pCMV-coat plasmid are transfected into the GFP expressing cell lines using calcium phosphate and the cells are incubated at a permissive temperature (e.g., 30° C., 34° C.) for 48 hr. By FACS analysis and sorting, those cells that no longer express GFP (or express significantly reduced levels) are isolated or “sorted” from the remaining GFP-positive cells and re-plated at the non-permissive temperature of 40° C. This isolated population of cells has been transfected with pCMV-coat plasmid that expresses functional R17/MS2 coat protein at the permissive temperature. After 24-48 hr at 40° C., the cells expressing GFP are isolated by FACS. This population of cells contains plasmid with the desired ts coat protein gene (e.g., no longer binds to operator at non-permissive temperature), and plasmid containing this modified ts coat protein gene is then re-isolated by Hirt extraction and re-transformation into bacteria. Plasmid is isolated from the bacteria without prior cloning and again subjected to the above procedure. Sequencing is performed on clonal ts coat protein genes and the desired mutant gene is then re-cloned into a pCMV-Script backbone that had not been subjected to mutagenesis. This construct then may be used for further recombinant protein application. It should be noted that selection for temperature sensitivity may also be performed in a similar manner, but with the temperatures switched. Thus, rather than having a heat sensitive coat protein with elevated temperatures being non-permissive, the ts coat protein would be cold sensitive, with lower temperatures (e.g., 30° C., 34° C.) being non-permissive.

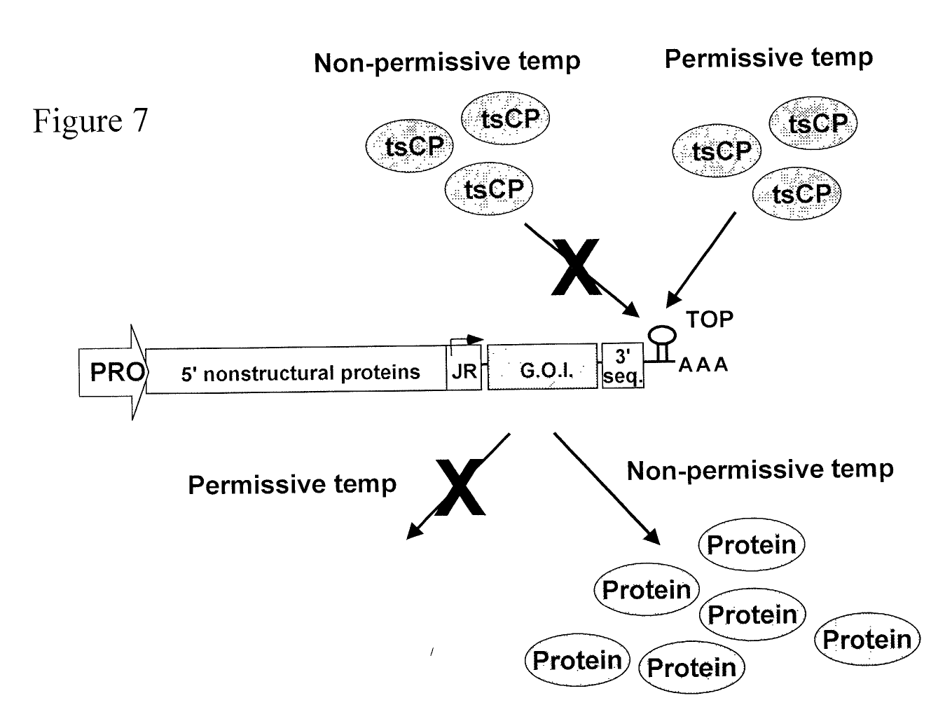

The ts coat protein cassette described above is next stably transfected into the desired cell line for recombinant protein expression (e.g., BHK, CHO, VERO), and the cells are subjected to G418 selection. Positive transformants are identified by transient transfection with plasmid pR17GFP and observing for differential GFP expression at permissive and non-permissive temperatures. This cell line is then used as the parental cell line source for incorporation of a DNA based alphavirus replicon (eukaryotic layered vector initiation system), as described above, that further comprises one or more R17/MS2 operator sequences and a heterologous gene to be expressed (FIG. 7). As described in example 3, a modified alphavirus replicon may be constructed by using the SINCPpuro construct as starting material. Incorporation of TOP sequences into the 3′-end is performed by overlapping PCR, using the following primer pairs in the first set of amplifications:

Primer pair #1
SIN3′ NOTfwd:

(SEQ ID NO: 36)

5′-TCTAGAGCGGCCGCCGCTACGCCCCAATG
SIN3′ TOPrev:

(SEQ ID NO: 37)

5′-AATTACATGGGTGATCCTCATGTTTTTGTTGATTAATAAAAGAAATA
Primer pair #2
SIN3′ TOPfwd:

(SEQ ID NO: 38)

5′-AAACATGAGGATCACCCATGTAATTTTGTTTTTAACATTTCAAAAAA

AA

SINBSSrev:

(SEQ ID NO: 39)

5′-AGGCTCAAGGCGCGCATGCCCGAC

Following amplification, the PCR products are purified using QIAquick, combined, and subjected to a second round of PCR amplification using the SIN3′NOTfwd and SINBSSrev primers. The resulting product, which contains the TOP sequence, is digested with NotI and BssHII, purified, and ligated into plasmid SINCPpuro that has also been digested with NotI and BssHII, and purified from an agarose gel. This DNA-based SIN replicon is designated SINCPpuroTOP. Heterologous sequences to be expressed may be inserted anywhere between the XhoI and NotI sites, and those constructs stably transformed into the desired ts coat protein expressing cell lines using puromycin selection. Following growth at a temperature permissive for coat protein function, recombinant protein expression is induced by shifting the cells to a temperature non-permissive for coat protein function.

### Example 5

### Generation of Alphavirus Replicon Particle Producer Cell Lines

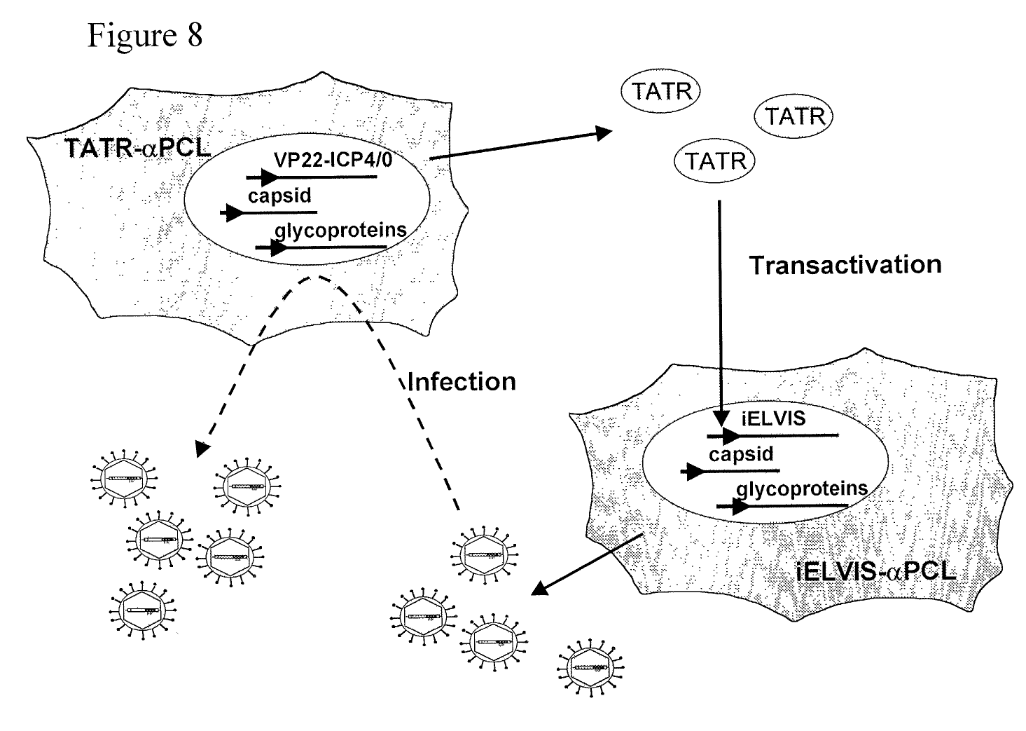

This example describes an Alphavirus Replicon Particle Producer Cell Line (ARP-PCL) for use in producing alphavirus replicon particles. The ARP-PCL is an entirely cell-based system that is used to produce alphavirus replicon particles that are free from contaminating replication competent virus (FIG. 8). As such, this system does not require transient transfection approaches to generate alphavirus vector particles.

Briefly, generation of ARP-PCL can be initiated from any desired parent cell line (e.g., BHK, CHO, Vero). The first step necessary for developing an ARP-PCL is to derive an alphavirus replicon packaging cell line (PCL). The process for constructing an alphavirus replicon PCL is well described in U.S. Pat. Nos. 4,789,245 and 5,843,723, and also WO 9738087 and WO 9918226 (each incorporated herein by reference). The second required step is to derive two new cell lines, beginning with the alphavirus replicon PCL as starting material. The first of the two new cell lines is derived by stably transforming the alphavirus replicon PCL with an expression cassette encoding a “transactivator-transporter fusion protein”. This cell line is known as TATR-αPCL. The second of the two new cell lines is derived by stably transforming the alphavirus replicon PCL with an expression cassette corresponding to an alphavirus-derived Eukaryotic Layered Vector Initiation System (ELVIS). Derivation of ELVIS is described in U.S. Pat. Nos. 5,814,482 and 6,015,686 (incorporated herein by reference). The ELVIS vector is constructed to contain a 5′ promoter that is activated in trans (trans-activated) by the transactivator-transporter fusion protein. Additionally, the ELVIS vector further includes the heterologous gene of interest to be expressed by the packaged replicons. The second cell line is known as iELVIS-αPCL. To produce alphavirus replicon particles, the iELVIS-αPCL is grown in culture to a desired density. In the second step, the TATR-αPCL cell line is mixed with the culture. The transactivator-transporter fusion protein expressed from the TATR-αPCL cell line enters the iELVIS-αPCL cell line, resulting in the induction of the ELVIS vector, which in turn results in the induction of alphavirus structural protein synthesis and the production of replicon particles. The replicon particles in turn will infect remaining cells in the culture not already undergoing alphavirus nonstructural protein-catalyzed biosynthesis, resulting in the production of replicon particles from all cells in the culture. The time and relative proportion in a culture of the TATR-αPCL and iELVIS-αPCL cell lines can be varied for optimal replicon particle production.

### Construction of TATR-αPCL cell line

The TATR-αPCL cell line (FIG. 8) is constructed by stably transforming the alphavirus replicon PCL with an expression cassette encoding the transactivator-transporter fusion protein (TATR). Alternatively, the TATR expression cassette can be inserted first into a desired parent cell line (e.g. BHK, CHO, Vero) prior to introduction of the alphavirus structural protein expression cassettes. In preferred embodiments, the transactivator can be the infected cell protein (ICP) 0 or 4 (ICP0, ICP4) from herpes simplex virus (HSV-1), and the transporter VP22, the product of the UL49 gene of HSV-1. As an example, construction of a functional TATR expression cassette plasmid can include the following ordered elements: Promoter/intron (e.g. CMV immediate early/intron A-ICPO (or ICP4)/VP22 in-frame fusion-polyadenylation/transcription termination sequence. This plasmid is known as pTATR. Plasmid pTATR can also include an expression cassette encoding a selectable drug-resistance enzyme. Stable introduction of pTATR into the PCL cell line is accomplished by transfection and isolation of individual cell clones under positive drug selection, using methods common to those skilled in the art. This cell line is known as TATR-αPCL.

Alternatively, in another embodiment, the transactivator-transporter fusion protein expression cassette can be composed of the following ordered elements: Promoter/intron (e.g. CMV immediate earlylintron A-activation domain (AD) of a transactivating protein (e.g., HSV-1 VP16)/α-complementing region of β-galactosidase geneNP22 in-frame fusion-polyadenylation/transcription termination sequence. These plasmids are collectively known as pADαTR. Stable introduction of pADαTR into the PCL cell line is accomplished by transfection and isolation of individual cell clones under positive drug selection, using methods common to those skilled in the art. This cell line is known as ADαTR-αPCL.

### Construction of iELVIS-αPCL cell line

The iELVIS-αPCL cell line (FIG. 8) is constructed by stably transforming the PCL cell line with a eukaryotic layered vector initiation system expression cassette encoding a heterologous gene of interest. A 5′ RNA polymerase II (pol II) promoter functionally linked to the alphavirus replicon cDNA is inactive in the PCL cell line or parent cell line, and can be only activated by introduction of a transactivating factor (i.e., transactivator-transporter fusion protein) into the cell. For example, in one embodiment, the ICP 8 promoter from HSV-1 is functionally linked to the desired alphavirus replicon cDNA to generate the ELVIS vector. This plasmid is known as piELVIS. The HSV-1 ICP8 promoter is optimally transactivated with both ICPO and ICP4 proteins, but is also transactivated with either protein individually. Stable introduction of piELVIS into the PCL cell line is accomplished by transfection and isolation of individual cell clones under positive drug selection, using methods common to those skilled in the art. This cell line is known as iELVIS-αPCL.

Alternatively, in another embodiment, an ordered assembly consisting of several tandem DNA binding domains (DNA-BD) of Gal 4 (e.g. 5) followed in sequence by a TATA box is juxtaposed precisely upstream of the alphavirus replicon cDNA such that transcription in vivo initiates at the nucleotide corresponding to the authentic alphavirus 5′ end. This plasmid is known as pGAL4-ELVIS. A second expression plasmid encoding a fusion protein consisting of the cognate region of the 1-galactosidase recovered by α-complementation and the GAL 4 DNA binding domain. This plasmid is known as pβDBD. Stable introduction of plasmids pGAL4-ELVIS and pβDBD into the PCL cell line is accomplished by transfection and isolation of individual cell clones under positive drug selections, using methods common to those skilled in the art. This cell line is known as GAL4-ELVIS-αPCL.

Production of functional alphavirus replicon particles is accomplished by co-cultivation of either of the two following pairs of cell lines described in this example:

1. TATR-αPCL and iELVIS-αPCL

2. ADαTR-αPCL and pGAL4-ELVIS-αPCL

### Construction of an ARP-PCL Using a Single Cell Line

Alternatively, an ARP-PCL that uses only a single cell line to produce alphavirus replicon particles can be constructed. In one embodiment, tandem repeats of the translational operator (TOP) sequence, which is the target binding sequence of the R17/MS2 bacteriophage coat protein (CP, described in Example 4), is inserted into a DNA-based alphavirus replicon or ELVIS vector as described above. This plasmid is known as pELVIS2TOP. Stable introduction of plasmids pELVIS2TOP and a ts coat protein expression cassette (described in Example 4) into the PCL cell line is accomplished by transfection and isolation of individual cell clones under positive drug selections, using the teaching provided herein and methods common to those skilled in the art. This cell line is known as ELVISTOP-αPCL. Induction of the ELVISTOP-αPCL cell line and production of alphavirus replicon particles is accomplished by shifting the culture conditions to a temperature that is non-permissive for coat protein function.

### Example 6

### Use of Alphavirus Replicons to Identify Differentially Expressed Genes

This example describes a method for using alphavirus replicons to identify differentially expressed genes between normal tissue, and its primary tumor and metastatic derivatives. The first step in this procedure is to generate uncloned double stranded cDNA libraries, starting with RNA, which can alternatively be polyA-selected, from normal tissue, and its primary tumor and metastatic derivatives. As an example, and is common to those skilled in the art, the 5′ ends of primers used for first strand and second strand cDNA synthesis can be modified to facilitate cloning into the cDNA of an alphavirus replicon vector. Insertion of the cDNA can use desired restriction sites, or alternatively, other approaches, such as the Gateway system, avoiding intra-gene restriction endonuclease digestion. As an example, to identify differentially expressed genes in cells from a primary tumor, compared to cells from normal tissue in the same individual, the cDNA generated from the primary tumor cell is inserted into the alphavirus replicon cDNA in a “sense” orientation, which corresponds to direction in which the message RNA is translated in the cell. Secondly, the cDNA generated from cells of normal tissue is inserted into the alphavirus replicon cDNA in an “anti-sense” orientation, which corresponds to the opposite direction in which the message RNA is translated in the cell. These cDNA libraries are referred to as Alpha+PTlib and AlphaNT−. The Alpha+PTlib and AlphaNT−cDNA libraries are linearized, transcribed in vitro, and the reactions are treated with DNase, and the single-stranded RNA products are purified by, for example, G-50 Sephadex chromatography. The purified Alpha+PTlib and AlphaNT− in vitro transcribed RNAs are allowed to hybridize, under conditions common to those skilled in the art. Subsequently, the non-hybridized single-stranded RNAs (ssRNA) are separated from the double-stranded (dsRNA) hybridized RNAs by hydroxy-apatite chromatography. The selected ssRNA can be further purified by an additional hydroxy-apatite chromatography step. Alternatively, the dsRNA can be degraded by dsRNA-specific RNases, resulting in a selected ssRNA library pool. The ssRNA pool selected by either of these methods can be re-hybridized with the in vitro transcribed AlphaNT−cDNA library, to increase the purity of isolation of unique RNAs expressed in cells from primary tumor. The ssRNA from the second round of hybridization is purified, as described above. The ssRNA pool corresponding to RNAs that are differentially expressed in tumor cells can be amplified by electroporation into the alphavirus replicon packaging cell line (αPCL), described in Example 6, resulting in a replicon particle library.

Alternatively, the alphavirus replicon can be electroporated into the Attention:PCL, diluted into 1% agarose equilibrated to 40° C., then added to an αPCL monolayer. If the electorporated αPCL is diluted suitably, individual plaques are visible within 48 hrs. These individual plaques contain a small stock of replicon particles corresponding to a single RNA expressed differentially in the cells of a primary tumor, compared to normal tissue. The replicon particles can be amplified further by infected a fresh αPCL monolayer. The sequence of the differentially expressed RNA corresponding to each plaque can be determined using methods common to those skilled in the art. Additionally, the replicon particle stocks can be used directly in various gene function cell-based assays.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated nucleic acid molecule, comprising an alphavirus nonstructural protein gene which, when operably incorporated into an alphavirus replicon particle, has a reduced level of vector-specific RNA synthesis, as compared to the wild-type replicon particle, and the same or greater level of proteins encoded by RNA transcribed from the subgenomic junction region promoter, as compared to the wild-type alphavirus replicon particle, and wherein said alphavirus nonstructural protein gene encodes a protein with a substitution in an amino acid residue of nsP2 selected from the group consisting of residues 1, 10, 468, 469, 472, 708, 712, 713, and 721.

2. An isolated nucleic acid molecule, comprising an alphavirus nonstructural protein gene which, when operably incorporated into an alphavirus replicon particle, increases the time required to reach 50% inhibition of host-cell directed macromolecular synthesis following expression in mammalian cells, as compared to the wild-type alphavirus replicon particle, and wherein said alphavirus nonstructural protein gene encodes a protein with a substitution in an amino acid residue of nsP2 selected from the group consisting of residues 1, 10, 468, 469, 472, 708, 712, 713, and 721.

3. An isolated nucleic acid molecule, comprising an alphavirus nonstructural protein gene which, when operably incorporated into an alphavirus RNA vector replicon, alphavirus replicon particle, or eukaryotic layered vector initiation system, results in a vector capable of persistent replication following introduction into a mammalian cell, and wherein said alphavirus nonstructural protein gene encodes a protein with a substitution in an amino acid residue of nsP2 selected from the group consisting of residue 1, 10, 468, 469, 472, 708, 712, 713, and 721.

4. The isolated nucleic acid molecule according to claims 1, 2, or 3, wherein said alphavirus is Sindbis virus.

5. The isolated nucleic acid molecule according to claims 1, 2, or 3, wherein said alphavirus is Semliki Forest virus.

6. The isolated nucleic acid molecule according to claims 1, 2, or 3, wherein said alphavirus is Ross River virus.

7. The isolated nucleic acid molecule according to claims 1, 2, or 3, wherein said alphavirus is Venezuelan equine encephalitis virus.

8. The isolated nucleic acid molecule according to claims 1, 2, or 3, wherein said alphavirus is S.A.AR86 virus.

9. An alphavirus vector construct, comprising a 5′ promoter which initiates synthesis of viral RNA in vitro from cDNA, a 5′ sequence which initiates transcription of alphavirus RNA, a nucleic acid molecule which operably encodes all four alphaviral nonstructural proteins, including a nucleic acid molecule according to claims 1, 2, or 3, an alphavirus RNA polymerase recognition sequence and a 3′ polyadenylate tract.

10. An alphavirus RNA vector replicon capable of translation in a eukaryotic cell, comprising a 5′ sequence which initiates transcription of alphavirus RNA, a nucleic acid molecule which operably encodes all four alphaviral nonstructural proteins, including a nucleic acid molecule according to claims 1, 2, or 3, an alphavirus RNA polymerase recognition sequence and a 3′ polyadenylate tract.

11. A eukaryotic layered vector initiation system, comprising a 5′ promoter capable of initiating in vivo the 5′ synthesis of alphavirus RNA from cDNA, a sequence which initiates transcription of alphavirus RNA following the 5′ promoter, a nucleic acid molecule which operably encodes all four alphaviral nonstructural proteins, including a nucleic acid molecule according to claims 1, 2, or 3, an alphavirus RNA polymerase recognition sequence, and a 3′ polyadenylate tract.

12. The alphavirus vector construct, RNA vector replicon, or eukaryotic layered vector initiation system according to claims 9, 10, or 11, wherein said alphavirus is Sindbis virus.

13. The alphavirus vector construct, RNA vector replicon, or eukaryotic layered vector initiation system according to claims 9, 10, or 11, wherein said alphavirus is Semliki Forest virus.

14. The alphavirus vector construct, RNA vector replicon, or eukaryotic layered vector initiation system according to claims 9, 10, or 11, wherein said alphavirus is Ross River virus.

15. The alphavirus vector construct, RNA vector replicon, or eukaryotic layered vector initiation system according to claims 9, 10, or 11, wherein said alphavirus is Venezuelan equine encephalitis virus.

16. The alphavirus vector construct, RNA vector replicon, or eukaryotic layered vector initiation system according to claims 9, 10, or 11, wherein said alphavirus is S.A.AR86 virus.

17. An alphavirus replicon particle, comprising one or more alphavirus structural proteins, a lipid envelope, and an RNA vector replicon according to claim 10.

18. The alphavirus replicon particle according to claim 17, wherein said alphavirus structural protein and RNA vector replicon are derived from different alphavirus species.

19. A pharmaceutical composition, comprising an alphavirus RNA vector replicon according to claim 10, a eukaryotic layered vector initiation system according to claim 11, or an alphavirus replicon particle according to claim 17, in combination with a pharmaceutically acceptable carrier or diluent.

20. A host cell which contains an alphavirus vector construct according to claim 9, an alphavirus RNA vector replicon according to claim 10, a eukaryotic layered vector initiation system according to claim 11, or an alphavirus replicon particle according to claim 17.

21. A method for delivering a selected heterologous sequence to a vertebrate or insect cell, comprising administering to a vertebrate or insect cell an alphavirus vector construct according to claim 9, an alphavirus RNA vector replicon according to claim 10, an alphavirus replicon particle according to claim 17, or a eukaryotic layered vector initiation system according to claim 11.

22. A method of making alphavirus replicon particles, comprising:
(a) introducing a vector selected from the group consisting of a eukaryotic layered vector initiation system according to claim 11, an alphavirus RNA vector replicon according to claim 10, and an alphavirus replicon particle according to claim 17, into a packaging cell, under conditions and for a time sufficient to permit production of alphavirus replicon particles; and
(b) harvesting alphavirus replicon particles.

23. A method of making a selected protein, comprising introducing a vector which encodes a selected heterologous protein into a cell, said vector selected from the group consisting of a eukaryotic layered vector initiation system according to claim 11, an alphavirus RNA vector replicon according to claim 10, and an alphavirus replicon particle according to claim 17 and growing said cell under conditions and for a time sufficient to permit production of said selected protein.

24. The method according to claim 23, wherein said cell is a packaging cell.

25. The method according to claim 23, further comprising the step of harvesting protein from said cell.

26. The method according to claim 23, wherein said protein is selected from the group consisting of erythropoietin, basic FGF, factor VIII, VEGF, and t-PA.

27. An alphavirus producer cell line, comprising a cell containing one or more stably transformed alphavirus structural protein expression cassettes, and an alphavirus vector selected from the group consisting of an alphavirus vector construct according to claim 9, an alphavirus RNA vector replicon according to claim 10, and a eukaryotic layered vector initiation system according to claim 11.

28. An expression cassette, comprising a promoter operably linked to a nucleic acid molecule, said nucleic acid molecule encoding a temperature sensitive R17 coat protein.

29. A cell comprising an expression cassette according to claim 28.

30. The cell according to claim 29, further comprising an alphavirus vector selected from the group consisting of an alphavirus vector construct, an alphavirus RNA vector replicon, and a eukaryotic layered vector initiation system.

31. The cell according to claim 30, wherein said alphavirus vector contains a nucleic acid molecule according to claims 1, 2, or 3.

32. The cell according to claim 30, wherein said alphavirus vector further comprises an R17 translational operator site.

33. The cell according to claim 30, wherein said alphavirus vector further comprises a heterologous sequence.

34. The cell according to claim 30, further comprising an alphavirus structural protein expression cassette.

35. A method of making a selected recombinant protein, comprising:
(a) introducing into a cell an alphavirus vector encoding said recombinant protein, wherein said alphavirus vector is selected from the group consisting of a eukaryotic layered vector initiation system, an alphavirus vector construct, and an alphavirus RNA vector replicon, and wherein said alphavirus vector further comprising a ligand binding sequence;
(b) providing said cell with an expression cassette comprising a promoter operably linked to a nucleic acid molecule, said nucleic acid molecule encoding a temperature sensitive ligand;
(c) propagating the population of cells at a temperature permissive for binding of said temperature sensitive ligand to said ligand binding sequence; and
(d) shifting the population of cells to a temperature non-permissive for binding of said ligand to said ligand binding sequence, under conditions and for a time sufficient to permit production of the recombinant protein.


SEQUENCE LISTING (39 sequences)

SEQ ID NO: 1 - DNA, 35 bp, Artificial Sequence
  Recombinant DNA
atatactcgagaccatgattgaacaagatggattg

SEQ ID NO: 2 - DNA, 35 bp, Artificial Sequence
  Recombinant DNA
tatatagcggccgctcagaagaactcgtcaagaag

SEQ ID NO: 3 - DNA, 38 bp, Artificial Sequence
  Recombinant DNA
atataggatccttcgcatgattgaacaagatggattgc

SEQ ID NO: 4 - DNA, 36 bp, Artificial Sequence
  Recombinant DNA
atataggatcctcagaagaactcgtcaagaaggcga

SEQ ID NO: 5 - DNA, 28 bp, Artificial Sequence
  Recombinant DNA
attataagcttggctccaactccatctc

SEQ ID NO: 6 - DNA, 48 bp, Artificial Sequence
  Recombinant DNA
tatatgggcccgatttaggtgacactatagattgacggcgtagtacac

SEQ ID NO: 7 - DNA, 34 bp, Artificial Sequence
  Recombinant DNA
tatatggatccctcagtcttagcacgtcggcctc

SEQ ID NO: 8 - DNA, 30 bp, Artificial Sequence
  Recombinant DNA
atatatctcgaggtattcagtcctcctgct

SEQ ID NO: 9 - DNA, 30 bp, Artificial Sequence
  Recombinant DNA
attatggatccggcattagttgaaaccccg

SEQ ID NO: 10 - DNA, 35 bp, Artificial Sequence
  Recombinant DNA
tatatggtaccatgcaaaggcacggcaacgttttg

SEQ ID NO: 11 - DNA, 27 bp, Artificial Sequence
  Recombinant DNA
gaaatgttaaaaacaaaattttgttga

SEQ ID NO: 12 - DNA, 29 bp, Artificial Sequence
  Recombinant DNA
tatatgaattcgcgccgtcataccgcacc

SEQ ID NO: 13 - DNA, 29 bp, Artificial Sequence
  Recombinant DNA
atatacggagaacctgcgtgcaatccatc

SEQ ID NO: 14 - DNA, 29 bp, Artificial Sequence
  Recombinant DNA
atatactactactgtagtcttatatggtg

SEQ ID NO: 15 - DNA, 28 bp, Artificial Sequence
  Recombinant DNA
atataggagactgacaaagacacactca

SEQ ID NO: 16 - DNA, 29 bp, Artificial Sequence
  Recombinant DNA
atataggcctgatcttcagcccttcgtag

SEQ ID NO: 17 - DNA, 28 bp, Artificial Sequence
  Recombinant DNA
atataccaagcatctgcagctcatggcg

SEQ ID NO: 18 - DNA, 28 bp, Artificial Sequence
  Recombinant DNA
atatagttggtgggagagctaaccaacc

SEQ ID NO: 19 - DNA, 28 bp, Artificial Sequence
  Recombinant DNA
atatacgacacactgctggtagtggtgg

SEQ ID NO: 20 - DNA, 27 bp, Artificial Sequence
  Recombinant DNA
atatagctctcttcgggcgcggtggag

SEQ ID NO: 21 - DNA, 29 bp, Artificial Sequence
  Recombinant DNA
atataggcaggttcgacttggtctttgtg

SEQ ID NO: 22 - DNA, 29 bp, Artificial Sequence
  Recombinant DNA
atatacggagaacctgcgtgcaatccatc

SEQ ID NO: 23 - DNA, 30 bp, Artificial Sequence
  Recombinant DNA
atatagatgtgcaccctgaaccccgcagac

SEQ ID NO: 24 - DNA, 36 bp, Artificial Sequence
  Recombinant DNA
tattacccgggtgcctacatattgggtgagaccatg

SEQ ID NO: 25 - DNA, 37 bp, Artificial Sequence
  Recombinant DNA
tattacccgggtgcgcactcgatcaagtcgagtagtg

SEQ ID NO: 26 - DNA, 25 bp, Artificial Sequence
  Recombinant DNA
ctatccgacagtagcatcttatcag

SEQ ID NO: 27 - DNA, 22 bp, Artificial Sequence
  Recombinant DNA
gtcgcctgcttgaagtgttctg

SEQ ID NO: 28 - DNA, 23 bp, Artificial Sequence
  Recombinant DNA
gaagccattgacatgaggacggc

SEQ ID NO: 29 - DNA, 22 bp, Artificial Sequence
  Recombinant DNA
ctgcgggttcagggtgtacgtc

SEQ ID NO: 30 - DNA, 22 bp, Artificial Sequence
  Recombinant DNA
atgtctggttcgtctggcccac

SEQ ID NO: 31 - DNA, 25 bp, Artificial Sequence
  Recombinant DNA
ctctatcaacttcttggccttgaag

SEQ ID NO: 32 - DNA, 35 bp, Artificial Sequence
  Recombinant DNA
atatatggatccatggcttctaactttactcagtt

SEQ ID NO: 33 - DNA, 35 bp, Artificial Sequence
  Recombinant DNA
atatataagcttttagtagatgccggagtttgctg

SEQ ID NO: 34 - DNA, 53 bp, Artificial Sequence
  Recombinant DNA
atatatgctagccatgaggatcacccatggtcgccaccatggtgagcaagggc

SEQ ID NO: 35 - DNA, 31 bp, Artificial Sequence
  Recombinant DNA
atatattctagagtcgcggccgcgcatctac

SEQ ID NO: 36 - DNA, 29 bp, Artificial Sequence
  Recombinant DNA
tctagagcggccgccgctacgccccaatg

SEQ ID NO: 37 - DNA, 47 bp, Artificial Sequence
  Recombinant DNA
aattacatgggtgatcctcatgtttttgttgattaataaaagaaata

SEQ ID NO: 38 - DNA, 49 bp, Artificial Sequence
  Recombinant DNA
aaacatgaggatcacccatgtaattttgtttttaacatttcaaaaaaaa

SEQ ID NO: 39 - DNA, 24 bp, Artificial Sequence
  Recombinant DNA
aggctcaaggcgcgcatgcccgac